SEQUENCE LISTING (91 sequences)

SEQ ID NO: 1 - protein, 76 aa, Homo sapiens [sequence omitted]

SEQ ID NO: 2 - protein, 91 aa, Homo sapiens
EVPQLTDLSFVDITDSSIGLRWTPLNSSTIIGYRITVVAAGEGIPIFEDFVDSSVGYYTVTGLEPGIDYDISVITLINGGESAPTTLTQQT

SEQ ID NO: 3 - protein, 3 aa, Artificial Sequence
  Artifically synthesized linker sequence
GIG

SEQ ID NO: 4 - protein, 5 aa, Artificial Sequence
  Artifically synthesized linker sequence
SGGGG

SEQ ID NO: 5 - protein, 7 aa, Artificial Sequence
  Artifically synthesized linker sequence
SGGGGIG

SEQ ID NO: 6 - protein, 12 aa, Artificial Sequence
  Artifically synthesized linker sequence
SGGGGSGGGGIG

SEQ ID NO: 7 - protein, 10 aa, Artificial Sequence
  Artifically synthesized linker sequence
SGGGGSGGGG

SEQ ID NO: 8 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 9 - protein, 188 aa, Homo sapiens [sequence omitted]

SEQ ID NO: 10 - protein, 165 aa, Homo sapiens [sequence omitted]

SEQ ID NO: 11 - protein, 165 aa, Homo sapiens
CDLPQTHSLGSRRTLMLLAQMRRISLFSCLKDRRDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 12 - protein, 189 aa, Homo sapiens
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 13 - protein, 189 aa, Homo sapiens
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 14 - protein, 189 aa, Homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 15 - protein, 189 aa, Homo sapiens
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 16 - protein, 190 aa, Mus musculus
MARLCAFLVMLIVMSYWSICSLGCDLPHTYNLRNKRALKVLAQMRRLPFLSCLKDRQDFGFPLEKVDNQQIQKAQAIPVLRDLTQQTLNLFTSKASSAAWNATLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 17 - protein, 192 aa, Rattus norvegicus
MARLCAFLMSLVVVSYWSACCLGCDLPHTHNLRNKRVFTLLAQMRRLSPVSCLKDRKYFGFPLEKVDGQQIQKAQAIPVLHELTQQILSLFTSKESSTAWDATLLDSFCNDLQQQLSGLQACLMQQVGVQESPLTQEDSLLAVREYFHRITVYLRENKHSPCAWEVVKAEVWRALSSSANLMGRLREERNES

SEQ ID NO: 18 - protein, 93 aa, Oryctolagus cuniculus
  misc_feature (44)..(44): Xaa can be any naturally occurring amino acid
SCLKDRKDFGFPLEKVDAQQIQKAQAISILHELSQQVLNIYTSXDSSAAWDATLLDSFCNDLQQQLSGLQACQMHQVGVQEPPLAQEDSLLAV

SEQ ID NO: 19 - protein, 155 aa, Artificial Sequence
  Artifically synthesized modified hetero-dimeric ubiquitin protein 1041-D11
MQIFVWTWTGKTITLEVEPSDTIENVKAKIQDKEGIPPDQQRLIWAGKQLEDGRTLSDYNIQRKFPLHLVLRLRGGGIGMRIFVTTQTGKTITLEVEPSDTIENVKAKIQDKEGIPPDQQRLIWAGKQLEDGRTLSDYNIWSNWELHLVLRLRAA

SEQ ID NO: 20 - protein, 155 aa, Artificial Sequence
  Artifically synthesized modified hetero-dimeric ubiquitin protein 1255-B9
MTIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 21 - protein, 155 aa, Artificial Sequence
  Artifically synthesized modified hetero-dimeric ubiquitin protein 1255-B10
MTIWVHTLTGKTITLEVEPSDTIENVKAKIQDKEGIPPDQQRLIWAGKQLEDGRTLSDYNINFKLSLHLVLRLRAAGIGMQIFVATPTGKTITLEVEPSDTIENVKAKIQDKEGIPPDQQRLIWAGKQLEDGRTLSDYNIGWQSPLHLVLRLRAA

SEQ ID NO: 22 - protein, 155 aa, Artificial Sequence
  Artifically synthesized modified hetero-dimeric ubiquitin protein 1247-G11
MTIWVHTLTGKTITLEVEPSDTIENVKAKIQDKEGIPPDQQRLIWAGKQLEDGRTLSDYNINFKLSLHLVLRLRAAGIGMQIFVRTHTGKTITLEVEPSDTIENVKAKIQDKEGIPPDQQRLIWAGKQLEDGRTLSDYNIGWQSPLHLVLRLRAA

SEQ ID NO: 23 - protein, 155 aa, Artificial Sequence
  Artifically synthesized modified hetero-dimeric ubiquitin protein 1255-G12
MTIWVHTLTGKTITLEVEPSDTIENVKAKIQDKEGIPPDQQRLIWAGKQLEDGRTLSDYNINFKLSLHLVLRLRAAGIGMQIFVYTYTGKTITLEVEPSDTIENVKAKIQDKEGIPPDQQRLIWAGKQLEDGRTLSDYNIWSGEFLHLVLRLRAA

SEQ ID NO: 24 - protein, 155 aa, Artificial Sequence
  Artifically synthesized modified hetero-dimeric ubiquitin protein 1247-F8
MTIWVHTLTGKTITLEVEPSDTIENVKAKIQDKEGIPPDQQRLIWAGKQLEDGRTLSDYNINFKLSLHLVLRLRAAGIGMQIFVLTHTGKTITLEVEPSDTIENVKAKIQDKEGIPPDQQRLIWAGKQLEDGRTLSDYNIWNKDWLHLVLRLRAA

SEQ ID NO: 25 - protein, 155 aa, Artificial Sequence
  Artifically synthesized modified hetero-dimeric ubiquitin protein 1237-B10
MTIWVHTLTGKTITLEVEPSDTIENVKAKIQDKEGIPPDQQRLIWAGKQLEDGRTLSDYNINFKLSLHLVLRLRAARIGMQIFVHTTTGKTITLEVEPSDTIENVKAKIQDKEGIPPDQQRLIWAGKQLEDGRTLSDYNIWHHDMLHLVLRLRAA

SEQ ID NO: 26 - protein, 155 aa, Artificial Sequence
  Artifically synthesized modified hetero-dimeric ubiquitin protein 1237-H4
MTIWVHTLTGKTITLEVEPSDTIENVKAKIQDKEGIPPDQQRLIWAGKQLEDGRTLSDYNINFKLSLHLVLRLRAAGIGMQIFVSTYTGKTITLEVEPSDTIENVKAKIQDKEGIPPDQQRLIWAGKQLADGRTLSDYNIWPGDMLHLVLRLRAA

SEQ ID NO: 27 - protein, 155 aa, Artificial Sequence
  Artifically synthesized modified hetero-dimeric ubiquitin protein 1239-B10
MTIWVHTLTGKTITLEVEPSDTIENVKAKIQDKEGIPPDQQRLIWAGKQLEDGRTLSDYNINFKLSLHLVLRLRAAGIGMQIFVDTPTGKTITLEVEPSDTIENVKAKIQDKKGIPPDQQRLIWAGKQLEDGRTLSDYNIGRLPKLHLVLRLRAA

SEQ ID NO: 28 - protein, 155 aa, Artificial Sequence
  Artifically synthesized modified hetero-dimeric ubiquitin protein 1246-H5
MTIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGYQAPLHLVLRLRAA

SEQ ID NO: 29 - protein, 155 aa, Artificial Sequence
  Artifically synthesized modified hetero-dimeric ubiquitin protein 1247-G1
MTIWVHTLTGKTITLEVEPSDTIENVKAKIQDKEGIPSDQQRLIWAGKQLEDGRTLSDYNINFKLSLHLVLRLRAAGIGMQIFVYTNTGKTITLEVEPSDTIENVKAKIQDKEGIPPDQQRLIWAGKQLEDGRTLSDYNIWQHDFLHLVLRLRAA

SEQ ID NO: 30 - protein, 155 aa, Artificial Sequence
  Artifically synthesized modified hetero-dimeric ubiquitin protein 1247-H2
MTIWVHTLTGKTITLEVEPSDTIENVKAKIQDKEGIPPDQQRLIWAGKQLEDGRTLSDYNINFKLSLHLVLRLRAAGIGMQIFVDTPTGKTITLEVEPSDTIENVKAKIQDKEGIPPDQQRLIWAGKQLEDGRTLSDYNIWHHDFLHLVLRLRAA

SEQ ID NO: 31 - protein, 155 aa, Artificial Sequence
  Artifically synthesized modified hetero-dimeric ubiquitin protein 1248-E1
MTIWVHTLTGKTITLEVEPSDTIENVKAKIQDKEGIPPDQQRLIWAGKQLEDGRTLSDYNINFKLSLHLVLRLRAAGIGMQIFVHTETGKTITLGVEPSDTIENVKAKIQDKEGIPPDQQRLIWAGKQLEDGRTLSDYNIGYQVPLHLVLRLRAA

SEQ ID NO: 32 - protein, 155 aa, Artificial Sequence
  Artifically synthesized modified hetero-dimeric ubiquitin protein 1249-E5
MTIWVHTLTGKTITLEVEPSDTIENVKAKIQDREGIPPDQQRLIWAGKQLEDGRTLSDYNINFKLSLHLVLRLRAAGIGMQIFVDTPTGKTITLEVEPSDTIENVKAKIQDKEGIPPDQQRLIWAGKQLEDERTLSDYNIDRLPVLHLVLRLRAA

SEQ ID NO: 33 - protein, 155 aa, Artificial Sequence
  Artifically synthesized modified hetero-dimeric ubiquitin protein 1253-A11
MTIWVHTLTGKTITLEVEPSDTIENVKAKIQDKEGIPPDQQRLIWAGKQLEDGRTLSDYNINFKLSLHLVLRLRAAGIGMQIFVSTATGKTITLEVEPSDTIENVKAKIQDKEGIPPDQQRLIWAGKQLEDGRTLSDYNIGWQAPLHLVLRLRAA

SEQ ID NO: 34 - protein, 155 aa, Artificial Sequence
  Artifically synthesized modified hetero-dimeric ubiquitin protein 1255-A8
MTIWVHTLTGKTITLEVEPSDTIENVKAKIQDKEGIPPDQQRLIWAGKQLEDGRTLSDYNINFKLSLHLVLRLRAAGIGMQIFVLTDTGKTITLEVEPSDTIENVKAKIQDKEGIPPDQQRLIWAGKQLEDGRTLSDYNIGWQSPLHLVLRLRAA

SEQ ID NO: 35 - protein, 155 aa, Artificial Sequence
  Artifically synthesized modified hetero-dimeric ubiquitin protein 1255-G3
MTIWVHTLTGKTITLEVEPSDTIENVKAKIQDKEGIPPDQQRLIWAGKQLEDGRTLSDYNINFKLSLHLVLRLRAAGIGMQIFVLTTTGKTITLEVEPSDTIENVKAKIQDKEGIPPDQQRLIWAGKQLEDGRTLSDYNIGWQSPLHLVLRLRAA

SEQ ID NO: 36 - protein, 155 aa, Artificial Sequence
  Artifically synthesized modified hetero-dimeric ubiquitin protein 1255-H3
MQIFVGTGTGKTITLEVEPSDTIENVKAKIQDKEGIPPDQQRLIWAGKQLEDGRTLSDYNINFKLSLHLVLRLRAAGIGMQIFVHTWTEKTITLEVEPSDTIENVKAKIQDKEGIPPDQQRLIWAGKQLEDGRTLSDYNIGYQAPLHLVLRLRAA

SEQ ID NO: 37 - protein, 326 aa, Artificial Sequence
  Artifically synthesized fusion protein IFN-1041-D11
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMQIFVWTWTGKTITLEVEPSDTIENVKAKIQDKEGIPPDQQRLIWAGKQLEDGRTLSDYNIQRKFPLHLVLRLRGGGIGMRIFVTTQTGKTITLEVEPSDTIENVKAKIQDKEGIPPDQQRLIWAGKQLEDGRTLSDYNIWSNWELHLVLRLRAA

SEQ ID NO: 38 - protein, 326 aa, Artificial Sequence
  Artifically synthesized fusion protein IFN-1255-B9
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 39 - protein, 326 aa, Artificial Sequence
  Artifically synthesized fusion protein IFN-1255-B10
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVATPTGKTITLEVEPSDTIENVKAKIQDKEGIPPDQQRLIWAGKQLEDGRTLSDYNIGWQSPLHLVLRLRAA

SEQ ID NO: 40 - protein, 326 aa, Artificial Sequence
  Artifically synthesized fusion protein IFN-1247-G11
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVRTHTGKTITLEVEPSDTIENVKAKIQDKEGIPPDQQRLIWAGKQLEDGRTLSDYNIGWQSPLHLVLRLRAA

SEQ ID NO: 41 - protein, 326 aa, Artificial Sequence
  Artifically synthesized fusion protein IFN-1255-G12
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVYTYTGKTITLEVEPSDTIENVKAKIQDKEGIPPDQQRLIWAGKQLEDGRTLSDYNIWSGEFLHLVLRLRAA

SEQ ID NO: 42 - protein, 326 aa, Artificial Sequence
  Artifically synthesized fusion protein IFN-1247-F8
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVLTHTGKTITLEVEPSDTIENVKAKIQDKEGIPPDQQRLIWAGKQLEDGRTLSDYNIWNKDWLHLVLRLRAA

SEQ ID NO: 43 - protein, 326 aa, Artificial Sequence
  Artifically synthesized fusion protein IFN-1237-B10
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAARIGMQIFVHTTTGKTITLEVEPSDTIENVKAKIQDKEGIPPDQQRLIWAGKQLEDGRTLSDYNIWHHDMLHLVLRLRAA

SEQ ID NO: 44 - protein, 326 aa, Artificial Sequence
  Artifically synthesized fusion protein IFN-1237-H4
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVSTYTGKTITLEVEPSDTIENVKAKIQDKEGIPPDQQRLIWAGKQLADGRTLSDYNIWPGDMLHLVLRLRAA

SEQ ID NO: 45 - protein, 326 aa, Artificial Sequence
  Artifically synthesized fusion protein IFN-1239-B10
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVDTPTGKTITLEVEPSDTIENVKAKIQDKKGIPPDQQRLIWAGKQLEDGRTLSDYNIGRLPKLHLVLRLRAA

SEQ ID NO: 46 - protein, 326 aa, Artificial Sequence
  Artifically synthesized fusion protein IFN-1246-H5
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGYQAPLHLVLRLRAA

SEQ ID NO: 47 - protein, 326 aa, Artificial Sequence [sequence omitted]

SEQ ID NO: 48 - protein, 326 aa, Artificial Sequence
  Artifically synthesized fusion protein IFN-1247-H2
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVDTPTGKTITLEVEPSDTIENVKAKIQDKEGIPPDQQRLIWAGKQLEDGRTLSDYNIWHHDFLHLVLRLRAA

SEQ ID NO: 49 - protein, 326 aa, Artificial Sequence
  Artifically synthesized fusion protein IFN-1248-E1
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVHTETGKTITLGVEPSDTIENVKAKIQDKEGIPPDQQRLIWAGKQLEDGRTLSDYNIGYQVPLHLVLRLRAA

SEQ ID NO: 50 - protein, 326 aa, Artificial Sequence
  Artifically synthesized fusion protein IFN-1249-E5
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDREGIPPDQQRLIWAGKQLEDGRTLSDYNINFKLSLHLVLRLRAAGIGMQIFVDTPTGKTITLEVEPSDTIENVKAKIQDKEGIPPDQQRLIWAGKQLEDERTLSDYNIDRLPVLHLVLRLRAA

SEQ ID NO: 51 - protein, 326 aa, Artificial Sequence
  Artifically synthesized fusion protein IFN-1253-A11
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVSTATGKTITLEVEPSDTIENVKAKIQDKEGIPPDQQRLIWAGKQLEDGRTLSDYNIGWQAPLHLVLRLRAA

SEQ ID NO: 52 - protein, 326 aa, Artificial Sequence
  Artifically synthesized fusion protein IFN-1255-A8
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVLTDTGKTITLEVEPSDTIENVKAKIQDKEGIPPDQQRLIWAGKQLEDGRTLSDYNIGWQSPLHLVLRLRAA

SEQ ID NO: 53 - protein, 326 aa, Artificial Sequence
  Artifically synthesized fusion protein IFN-1255-G3
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMTIWVHTLTGKTITLEVEPSDTIENVKAKIQDKEGIPPDQQRLIWAGKQLEDGRTLSDYNINFKLSLHLVLRLRAAGIGMQIFVLTTTGKTITLEVEPSDTIENVKAKIQDKEGIPPDQQRLIWAGKQLEDGRTLSDYNIGWQSPLHLVLRLRAA

SEQ ID NO: 54 - protein, 326 aa, Artificial Sequence
  Artifically synthesized fusion protein IFN-1255-H3
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMQIFVGTGTGKTITLEVEPSDTIENVKAKIQDKEGIPPDQQRLIWAGKQLEDGRTLSDYNINFKLSLHLVLRLRAAGIGMQIFVHTWTEKTITLEVEPSDTIENVKAKIQDKEGIPPDQQRLIWAGKQLEDGRTLSDYNIGYQAPLHLVLRLRAA

SEQ ID NO: 55 - protein, 325 aa, Artificial Sequence
  Artifically synthesized fusion protein 1041-D11-IFN
MQIFVWTWTGKTITLEVEPSDTIENVKAKIQDKEGIPPDQQRLIWAGKQLEDGRTLSDYNIQRKFPLHLVLRLRGGGIGMRIFVTTQTGKTITLEVEPSDTIENVKAKIQDKEGIPPDQQRLIWAGKQLEDGRTLSDYNIWSNWELHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 56 - protein, 325 aa, Artificial Sequence
  Artifically synthesized fusion protein 1255-B9-IFN
MTIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 57 - protein, 325 aa, Artificial Sequence [sequence omitted]

SEQ ID NO: 58 - protein, 325 aa, Artificial Sequence
  Artifically synthesized fusion protein 1247-G11-IFN
MTIWVHTLTGKTITLEVEPSDTIENVKAKIQDKEGIPPDQQRLIWAGKQLEDGRTLSDYNINFKLSLHLVLRLRAAGIGMQIFVRTHTGKTITLEVEPSDTIENVKAKIQDKEGIPPDQQRLIWAGKQLEDGRTLSDYNIGWQSPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 59 - protein, 325 aa, Artificial Sequence
  Artifically synthesized fusion protein 1255-G12-IFN
MTIWVHTLTGKTITLEVEPSDTIENVKAKIQDKEGIPPDQQRLIWAGKQLEDGRTLSDYNINFKLSLHLVLRLRAAGIGMQIFVYTYTGKTITLEVEPSDTIENVKAKIQDKEGIPPDQQRLIWAGKQLEDGRTLSDYNIWSGEFLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 60 - protein, 325 aa, Artificial Sequence
  Artifically synthesized fusion protein 1247-F8-IFN
MTIWVHTLTGKTITLEVEPSDTIENVKAKIQDKEGIPPDQQRLIWAGKQLEDGRTLSDYNINFKLSLHLVLRLRAAGIGMQIFVLTHTGKTITLEVEPSDTIENVKAKIQDKEGIPPDQQRLIWAGKQLEDGRTLSDYNIWNKDWLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 61 - protein, 325 aa, Artificial Sequence
  Artifically synthesized fusion protein 1237-B10-IFN
MTIWVHTLTGKTITLEVEPSDTIENVKAKIQDKEGIPPDQQRLIWAGKQLEDGRTLSDYNINFKLSLHLVLRLRAARIGMQIFVHTTTGKTITLEVEPSDTIENVKAKIQDKEGIPPDQQRLIWAGKQLEDGRTLSDYNIWHHDMLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 62 - protein, 325 aa, Artificial Sequence
  Artifically synthesized fusion protein 1237-H4-IFN
MTIWVHTLTGKTITLEVEPSDTIENVKAKIQDKEGIPPDQQRLIWAGKQLEDGRTLSDYNINFKLSLHLVLRLRAAGIGMQIFVSTYTGKTITLEVEPSDTIENVKAKIQDKEGIPPDQQRLIWAGKQLADGRTLSDYNIWPGDMLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 63 - protein, 325 aa, Artificial Sequence
  Artifically synthesized fusion protein 1239-B10-IFN
MTIWVHTLTGKTITLEVEPSDTIENVKAKIQDKEGIPPDQQRLIWAGKQLEDGRTLSDYNINFKLSLHLVLRLRAAGIGMQIFVDTPTGKTITLEVEPSDTIENVKAKIQDKKGIPPDQQRLIWAGKQLEDGRTLSDYNIGRLPKLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 64 - protein, 325 aa, Artificial Sequence
  Artifically synthesized fusion protein 1246-H5-IFN
MTIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGYQAPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 65 - protein, 325 aa, Artificial Sequence
  Artifically synthesized fusion protein 1247-G1-IFN
MTIWVHTLTGKTITLEVEPSDTIENVKAKIQDKEGIPSDQQRLIWAGKQLEDGRTLSDYNINFKLSLHLVLRLRAAGIGMQIFVYTNTGKTITLEVEPSDTIENVKAKIQDKEGIPPDQQRLIWAGKQLEDGRTLSDYNIWQHDFLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 66 - protein, 325 aa, Artificial Sequence
  Artifically synthesized fusion protein 1247-H2-IFN
MTIWVHTLTGKTITLEVEPSDTIENVKAKIQDKEGIPPDQQRLIWAGKQLEDGRTLSDYNINFKLSLHLVLRLRAAGIGMQIFVDTPTGKTITLEVEPSDTIENVKAKIQDKEGIPPDQQRLIWAGKQLEDGRTLSDYNIWHHDFLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 67 - protein, 325 aa, Artificial Sequence
  Artifically synthesized fusion protein 1248-E1-IFN
MTIWVHTLTGKTITLEVEPSDTIENVKAKIQDKEGIPPDQQRLIWAGKQLEDGRTLSDYNINFKLSLHLVLRLRAAGIGMQIFVHTETGKTITLGVEPSDTIENVKAKIQDKEGIPPDQQRLIWAGKQLEDGRTLSDYNIGYQVPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 68 - protein, 325 aa, Artificial Sequence
  Artifically synthesized fusion protein 1249-E5-IFN
MTIWVHTLTGKTITLEVEPSDTIENVKAKIQDREGIPPDQQRLIWAGKQLEDGRTLSDYNINFKLSLHLVLRLRAAGIGMQIFVDTPTGKTITLEVEPSDTIENVKAKIQDKEGIPPDQQRLIWAGKQLEDERTLSDYNIDRLPVLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 69 - protein, 325 aa, Artificial Sequence
  Artifically synthesized fusion protein 1253-A11-IFN
MTIWVHTLTGKTITLEVEPSDTIENVKAKIQDKEGIPPDQQRLIWAGKQLEDGRTLSDYNINFKLSLHLVLRLRAAGIGMQIFVSTATGKTITLEVEPSDTIENVKAKIQDKEGIPPDQQRLIWAGKQLEDGRTLSDYNIGWQAPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 70 - protein, 325 aa, Artificial Sequence
  Artifically synthesized fusion protein 1255-A8-IFN
MTIWVHTLTGKTITLEVEPSDTIENVKAKIQDKEGIPPDQQRLIWAGKQLEDGRTLSDYNINFKLSLHLVLRLRAAGIGMQIFVLTDTGKTITLEVEPSDTIENVKAKIQDKEGIPPDQQRLIWAGKQLEDGRTLSDYNIGWQSPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 71 - protein, 325 aa, Artificial Sequence
  Artifically synthesized fusion protein 1255-G3-IFN
MTIWVHTLTGKTITLEVEPSDTIENVKAKIQDKEGIPPDQQRLIWAGKQLEDGRTLSDYNINFKLSLHLVLRLRAAGIGMQIFVLTTTGKTITLEVEPSDTIENVKAKIQDKEGIPPDQQRLIWAGKQLEDGRTLSDYNIGWQSPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 72 - protein, 325 aa, Artificial Sequence
  Artifically synthesized fusion protein 1255-H3-IFN
MQIFVGTGTGKTITLEVEPSDTIENVKAKIQDKEGIPPDQQRLIWAGKQLEDGRTLSDYNINFKLSLHLVLRLRAAGIGMQIFVHTWTEKTITLEVEPSDTIENVKAKIQDKEGIPPDQQRLIWAGKQLEDGRTLSDYNIGYQAPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 73 - protein, 3 aa, Artificial Sequence
  Artifically synthesized linker sequence RIG
RIG

SEQ ID NO: 74 - protein, 155 aa, Artificial Sequence
  Artifically synthesized modified hetero-dimeric ubiquitin protein 1353-H6
MVIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 75 - protein, 155 aa, Artificial Sequence
  Artifically synthesized modified hetero-dimeric ubiquitin protein 1351-E9
MKIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 76 - protein, 155 aa, Artificial Sequence
  Artifically synthesized modified hetero-dimeric ubiquitin protein 1354-A8
MRIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 77 - protein, 155 aa, Artificial Sequence
  Artifically synthesized modified hetero-dimeric ubiquitin protein 1351-E9_F63P
MKIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 78 - protein, 326 aa, Artificial Sequence
  Artifically synthesized fusion protein IFN-1353-H6
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMVIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 79 - protein, 326 aa, Artificial Sequence
  Artifically synthesized fusion protein IFN-1351-E9
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMKIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 80 - protein, 326 aa, Artificial Sequence
  Artifically synthesized fusion protein IFN-1354-A8
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMRIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 81 - protein, 326 aa, Artificial Sequence
  Artifically synthesized fusion protein IFN-1351-E9_F63P
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGMKIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 82 - protein, 325 aa, Artificial Sequence
  Artifically synthesized fusion protein 1353-H6-IFN
MVIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 83 - protein, 325 aa, Artificial Sequence
  Artifically synthesized fusion protein 1351-E9-IFN
MKIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 84 - protein, 325 aa, Artificial Sequence
  Artifically synthesized fusion protein 1354-A8-IFN
MRIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 85 - protein, 325 aa, Artificial Sequence
  Artifically synthesized fusion protein 1351-E9_F63P-IFN
MKIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 86 - DNA, 15 bp, Artificial Sequence
  Artifically synthesized cis-acting element
tagtttcactttccc

SEQ ID NO: 87 - protein, 4 aa, Artificial Sequence
  Artifically synthesized basic linker sequence
GGGS

SEQ ID NO: 88 - protein, 4 aa, Artificial Sequence
  Artifically synthesized basic linker sequence
SGGG

SEQ ID NO: 89 - protein, 325 aa, Artificial Sequence
  Artifically synthesized fusion protein UB2-IFN
MQIFVKTLTGKTITLEVEPSDTIENVKAKIQDKEGIPPDQQRLIWAGKQLEDGRTLSDYNIQKESTLHLVLRLRAAGIGMQIFVKTLTGKTITLEVEPSDTIENVKAKIQDKEGIPPDQQRLIWAGKQLEDGRTLSDYNIQKESTLHLVLRLRAASGGGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 90 - protein, 326 aa, Artificial Sequence [sequence omitted]

SEQ ID NO: 91 - protein, 76 aa, Artificial Sequence
  Artifically synthesized ubiquitin mutein (F45W/G75A/G76A), start sequence for mutagenesis
MQIFVKTLTGKTITLEVEPSDTIENVKAKIQDKEGIPPDQQRLIWAGKQLEDGRTLSDYNIQKESTLHLVLRLRAA

(19) United States
(12) Patent Application Publication
(10) Pub. No.: US 2014/0219959 A1
(43) Pub. Date: Aug. 7, 2014

(54) HUMAN FUSION PROTEINS COMPRISING INTERFERONS AND TARGETED MODIFIED UBIQUITIN PROTEINS

(71) Applicants: Joerg Nerkamp, Halle/Saale (DE); Ilka Haenssgen, Halle/Saale (DE); Christian Lange, Halle/Saale (DE); Manja Gloser, Halle/Saale (DE); Arnd Steuemagel, Halle/Saale (DE); Antje Parther, Halle/Saale (DE)

(72) Inventors: Joerg Nerkamp, Halle/Saale (DE); Ilka Haenssgen, Halle/Saale (DE); Christian Lange, Halle/Saale (DE); Manja Gloser, Halle/Saale (DE); Arnd Steuemagel, Halle/Saale (DE); Antje Parther, Halle/Saale (DE)

(73) Assignee: Scil Proteins GmbH, Halle/Saale (DE)

(21) Appl. No.: 14/126,341

(22) PCT Filed: Jun. 15, 2012
(86) PCT No.: PCT/EP2012/061459

§ 371 Date: Dec. 13, 2013

(30) Foreign Application Priority Data
Jun. 15, 2011 (EP) PCT/EP2011/002962

(51) Int. Cl. A61K 38/21; A61K 45/06; C07K 14/555

(52) U.S. Cl. CPC A61K 38/212; C07K 14/555; A61K 45/06; C07K 2319/33
USPC 424/85.7; 530/351; 536/23.4; 435/320.1; 435/252.33; 435/69.7

(57) ABSTRACT

The present invention relates to fusion proteins in which a pharmaceutically active component is fused to an antibody mimetic. The invention specifically concerns fusion proteins comprising interferons or biologically active muteins thereof and modified hetero-dimeric ubiquitin proteins as specific targeting domain. The invention further relates to these fusion proteins for use in medicine, in particular for use in the treatment of cancer or infectious diseases. The invention is further directed to pharmaceutical compositions comprising a pharmaceutically acceptable carrier in combination with such fusion proteins, and in combination with cancer therapeutic agents. Moreover, the invention relates to a method for the generation of said fusion proteins.

The present invention relates to fusion proteins in which a pharmaceutically active component is fused directly or via a linker to an antibody mimetic. The invention specifically concerns fusion proteins comprising interferons or biologically active muteins thereof as the pharmaceutically active component and modified hetero-dimeric ubiquitin proteins as the antibody mimetic. The invention further relates to these fusion proteins for use in medicine, in particular for use in the treatment of cancer or infectious diseases. The invention also provides polynucleotides encoding such fusion proteins and vectors comprising such polynucleotides, as well as host cells comprising the aforementioned fusion proteins, polynucleotides, or vectors. The invention is further directed to pharmaceutical compositions comprising a pharmaceutically acceptable carrier in combination with such fusion proteins, polynucleotides, vectors or host cells. Moreover, the invention relates to a method for the generation of said fusion proteins.

### BACKGROUND OF THE INVENTION

There is a growing demand for binding molecules consisting of amino acids which are not immunoglobulins. While until now antibodies represent the best-established class of binding molecules there is still a need for new binding molecules in order to target ligands with high affinity and specificity since immunoglobulin molecules suffer from major drawbacks. Although antibodies can be produced quite easily and may be directed to almost any target, they have a quite complex molecular structure. There is an ongoing need to substitute antibodies by smaller molecules which can be handled in an easier way. These alternative binding agents can be beneficially used for instance in the medical fields of diagnosis, prophylaxis and treatment of diseases.

Proteins having relatively defined 3-dimensional structures, commonly referred to as protein scaffolds, may be used as starting material for the design of said alternative binding agents. These scaffolds typically contain one or more regions which are amenable to specific or random sequence variation, and such sequence randomisation is often carried out to produce a library of proteins from which the specific binding molecules may be selected. Molecules with a smaller size than antibodies and a comparable or even better affinity towards a target antigen are expected to be superior to antibodies in terms of pharmacokinetic properties and immunogenicity.

### Specific Targeting Proteins Based on Modified Ubiquitin

Ubiquitin is a small, monomeric, and cytosolic protein which is highly conserved in sequence and is present in all known eukaryotic cells from protozoans to vertebrates. The polypeptide chain of ubiquitin (see SEQ ID NO: 1) consists of 76 amino acids folded in an extraordinarily compact α/β structure (Vijay-Kumar et al., 1987 Apr. 5; J. Mol. Biol., 194(3):531-44): almost 87% of the polypeptide chain is involved in the formation of the secondary structural elements by means of hydrogen bonds. Secondary structures are three and a half alpha-helical turns as well as an antiparallel β sheet consisting of four strands. A further structural feature is a marked hydrophobic region in the protein interior between the alpha helix and the β sheet. Ubiquitin is a protein highly conserved among many species.

Because of its small size, artificial preparation of ubiquitin can be carried out both by chemical synthesis and by means of biotechnological methods. Due to the favourable folding properties, ubiquitin can be produced by genetic engineering using microorganisms such as Escherichia coli in relatively large amounts either in the cytosol or in the periplasmic space.

WO 04/106368 describes the generation of artificial binding proteins on the basis of ubiquitin proteins, also referred to as Affilin® (a registered trademark of Scil Proteins GmbH). WO 2011/073214 refers to a method for identifying multimeric ubiquitins with newly generated binding capability to a pre-defined ligand.

Compared to antibodies or other alternative scaffolds, artificial binding proteins on the basis of ubiquitin proteins have many advantages: high target affinity and specificity, small size, high stability, low immunogenicity, and cost effective manufacturing. Further, ubiquitin scaffolds are amenable to genetic and chemical modifications.

### Extra-Domain B of Fibronectin

Fibronectins (FN) are an important class of high molecular weight extracellular matrix glycoproteins abundantly expressed in healthy tissues and body fluids. Their main role consists in facilitating the adhesion of cells to a number of different extracellular matrices. The presence of fibronectins on the surface of non-transformed cells in culture as well as their absence in the case of transformed cells resulted in the identification of fibronectins as important adhesion proteins. They interact with numerous various other molecules, e.g. collagen, heparan sulphate-proteoglycans and fibrin and thus regulate the cell shape and the creation of the cytoskeleton. In addition, they are responsible for cell migration and cell differentiation during embryogenesis. They also play an important role in wound healing, in which they facilitate the migration of macrophages and other immune cells and in the formation of blood clots by enabling the adhesion of blood platelets to damaged regions of the blood vessels.

The extra-domain B (ED-B) of fibronectin is a small domain which is inserted by alternative splicing of the primary RNA transcript into the fibronectin molecule. The molecule is either present or omitted in fibronectin molecules of the extracellular matrix and represents a one of the most selective markers associated with angiogenesis and tissue remodelling, as it is abundantly expressed around new blood vessels, but undetectable in virtually all normal adult tissues (except for uterus and ovaries). ED-B is known to be involved primarily in cancer. High levels of ED-B expression were detected in primary lesions as well as metastatic sites of many human solid cancer entities, including breast, non-small cell lung, colorectal, pancreatic, human skin, hepatocellular, intracranial meningioma, glioblastoma (Menrad and Menssen, 2005 Expert Opin Ther Targets 9:491-500). Furthermore, ED-B can be bound to diagnostic agents and be favorably used as diagnostic tool. One example is its use in molecular imaging of atherosclerotic plaques and detection of cancer. Plenty of additional diagnostic uses are conceivable.

The amino acid sequence of 91 amino acids of human extra-domain B (ED-B) of fibronectin is shown in SEQ ID NO: 2. For expression of the protein, a start methionine has to be added. ED-B is abundant in mammals, e.g. in rodents, cattle, primates, carnivore, human etc. Examples of animals in which there is a 100% sequence identity to human ED-B are Rattus norvegicus, Bos taurus, Mus musculus, Equus caballus, Macaca mulatta, Canis lupus familiaris, and Pan troglodytes.

ED-B specifically accumulates in neo-vascular structures and represents a target for molecular intervention in cancer. A number of antibodies or antibody fragments to the ED-B domain of fibronectin are known in the art as potential therapeutics for cancer and other indications (see, for example, WO 97/45544, WO 07/054,120, WO 99/58570, WO 01/62800). A human single chain Fv antibody fragment specific to the ED-B domain of fibronectin has been verified to selectively target tumor neovasculature, both in experimental tumor models and in patients with cancer. Furthermore, conjugates comprising an anti-ED-B antibody or an anti-ED-B antibody fragment with IL-12, IL-2, IL-10, IL-15, IL-24, or GM-CSF have been described for targeting drugs for the manufacture of a medicament for inhibiting particularly cancer, angiogenesis, or neoplastic growth (see, for example, WO06/119897, W007/128,563, WO01/62298). The selective targeting of neovasculature of solid tumors with anti-ED-B antibodies or anti-ED-B antibody fragments conjugated to an appropriate effector function such as a cytotoxic or an immunostimulating agent has proven to be successful in animal experiments. For the therapy of pancreatic cancer, fusion proteins comprising an Interleukin-2 part and an anti-ED-B antibody part were combined with a small molecule (see, for example, WO 07/115,837).

WO 2011/073208 and WO 2011/073209 disclose multimeric proteins based on modified ubiquitin with high affinity binding to the extradomain B of fibronectin (ED-B). The applications describe anti-ED-B binding molecules showing a highly efficient targeting of tumor vasculature.

### Interferon

Interferon alpha (abbreviations: IFN-alpha or IFN-α) is a cytokine. More than 10 different subtypes encoded by different genes exist in human. All IFN-alpha subtypes, together with IFN-beta, bind to the IFN-alpha receptor (IFNAR), which is composed of two subunits, IFNAR1 and IFNAR2. IFNAR molecules are present on most cell types, making them responsive to IFN-alpha signals. Interactions between IFN-alpha and its receptor are highly species specific. For uses in medicine, especially interferons IFN-alpha 2a and IFN-alpha 2b are of interest. Both interferons show high affinity to the IFN-alpha receptor.

The role of interferon alpha has been studied in cancer. Medicaments containing IFN-alpha 2a or 2b were used initially for indications like Hairy cell leukemia and chronic myelogenous leukemia. IFN-alpha is still used in the treatment of renal cell carcinoma and cutaneous lymphoma but other therapies show improved efficacies compared to IFN-alpha. In order to obtain therapeutic responses, high doses of IFN-alpha have to be used, leading to high toxicity. IFN-alpha has many cellular effects including an anti-cancer activity and anti-viral activity. IFN-alpha therapy often has to be applied for many months in order to achieve a therapeutic result. Nevertheless, IFN-alpha is one of the very few cancer therapies that has the potential to have a curative effect on metastatic tumors in humans.

### TECHNICAL PROBLEMS UNDERLYING THE PRESENT INVENTION AND THEIR SOLUTION

Since cancer represents one of the leading causes for death worldwide, there is a growing need for improved agents for treating cancer. Current cancer therapeutic agents and radiation treatments suffer from poor selectivity. Most cancer therapeutic agents do not accumulate at the tumor site and thus fail to achieve adequate levels within the tumor. This results in significant side effects. Further, the toxicological profile of many cancer therapeutics limits dosing and thus their beneficial effect. Cancer therapeutic drugs, if given alone, often show poor tissue penetration and poor tumor uptake resulting in the accumulation of cancer therapeutic drugs in healthy tissue. Needless to say that there is a strong medical need to effectively treat cancer.

Innovative cancer treatments use tumor targeted delivery of anti-cancer drugs. These drugs should be directly targeted to the tumor and spare healthy tissue. Conjugates comprising a pharmaceutically active component and a binding protein (typically an antibody) which is directed against tumor antigens have been described in the prior art. However, these conjugates have drawbacks on the side of the pharmaceutically active component and/or on the side of the binding protein.

There remains a need in the art for conjugates in which the binding protein does not have the disadvantages of the commonly used antibodies as outlined above and in which the pharmaceutically active component exhibits an outstanding anti-tumor activity. In particular, there remains a strong need in the art for efficient tumor targeted therapeutics. Ideally, innovative conjugates in which the binding protein does not have the disadvantages of commonly used antibodies and in which the pharmaceutically active component exhibits an outstanding anti-tumor activity should be efficient therapeutics. In order to achieve this, the tumor target should be highly tumor specific and abundant in tumor tissue. Binding to a tumor target should occur with high affinity and selectivity. Further, in addition to high affinity target binding a tumor therapeutic should have a highly active functional domain employing a therapeutic effect.

It is thus an object of the present invention to provide novel fusion proteins comprising (i) targeted binding proteins that are advantageous as compared to antibodies and (ii) pharmaceutically active components that exhibit an improved anti-tumor activity.

An advantage associated with the fusion proteins of the present invention is a targeting of IFN-alpha to specific sites of disease, in particular cancer cells. Thereby, the efficacy of the treatment can be enhanced. Further, the specific curative effect on tumors is enhanced by directing the molecule to the specific cell. An advantage expected to be associated with fusion proteins of the present invention is therefore lower toxicity compared to IFN-alpha alone since doses of IFN-alpha are lower.

A further advantage of fusion proteins between IFN and an ED-B targeted binding protein might be that an anti-apoptotic effect might be beneficial in addressing tumors. The small size of the fusion protein is favorable for optimal penetration of a tumor.

Additional advantages associated with the fusion proteins of the present invention are an enhanced stability in plasma and an increased biological half-time in the body as compared to binding proteins without pharmaceutically active component (e.g. without an interferon part) and as compared to interferon without targeting domain. Extended plasma half-life and superior affinity to a disease target are beneficial for accumulation of the fusion protein in tumors. Without wishing to be bound by any particular theory, it is assumed that a reduced clearance of the fusion proteins of the invention causes the increased biological half-time.

The above-described objects are solved and the advantages are achieved by the subject-matter of the enclosed independent claims. Preferred embodiments of the invention are included in the dependent claims as well as in the following description, examples and figures.

The above overview does not necessarily describe all problems solved by the present invention.

### SUMMARY OF THE INVENTION

In a first aspect the present invention relates to a fusion protein comprising, essentially consisting of or consisting of the following parts: (i) an interferon or a biologically active mutein thereof; (ii) a modified hetero-dimeric ubiquitin protein that is capable of binding to a target molecule; and (iii) optionally a linker.

In a second aspect the present invention relates to the fusion protein according to the first aspect for use in medicine.

In a third aspect the present invention relates to the fusion protein according to the first aspect for use in the treatment of cancer or infectious diseases.

In a fourth aspect the present invention relates to a polynucleotide encoding the fusion protein as defined in the first aspect.

In a fifth aspect the present invention relates to a vector comprising the polynucleotide of the fourth aspect.

In a sixth aspect the present invention relates to a host cell comprising: a fusion protein as defined in the first aspect; a polynucleotide as defined in the fourth aspect; or a vector as defined in the fifth aspect.

In a seventh aspect the present invention relates to a pharmaceutical composition comprising: a fusion protein as defined in the first aspect; a polynucleotide as defined in the fourth aspect; a vector as defined in the fifth aspect; or a host cell as defined in the sixth aspect; and further comprising a pharmaceutically acceptable carrier.

In an eighth aspect the present invention relates to a method for generation of a fusion protein as defined in the first aspect, said method comprising the following steps:

(a) providing a population of differently modified dimeric ubiquitin proteins originating from monomeric ubiquitin proteins, said population comprising dimeric ubiquitin proteins comprising two modified ubiquitin monomers linked together, preferably in a head-to-tail arrangement, wherein each monomer of said dimeric protein is differently modified by substitutions of 1-8 amino acids of SEQ ID NO: 1 or SEQ ID NO: 91;
(b) providing a target molecule as potential ligand;
(c) contacting said population of differently modified proteins with said target molecule;
(d) identifying a modified dimeric ubiquitin protein by a screening process, wherein said modified dimeric ubiquitin protein binds to said target molecule with a specific binding affinity of Kd≦10⁻⁷ M;
(e) isolating said modified dimeric ubiquitin protein with said binding affinity; and
(f) fusing IFN-α or a biologically active mutein thereof to the modified dimeric ubiquitin protein obtained in step e).

In a ninth aspect the present invention relates to a method for the preparation of a fusion protein as defined in the first aspect, said method comprising the following steps:

(a) preparing a nucleic acid encoding a fusion protein as defined in the first aspect;
(b) introducing said nucleic acid into an expression vector;
(c) introducing said expression vector into a host cell;
(d) cultivating the host cell;
(e) subjecting the host cell to culturing conditions under which a fusion protein is expressed from said vector, thereby producing a fusion protein as defined in any one of claims 1 to 8; and
(f) optionally enriching or isolating the fusion protein produced in step (e).

This summary of the invention does not necessarily describe all features of the present invention. Other embodiments will become apparent from a review of the ensuing detailed description.

### BRIEF DESCRIPTION OF THE DRAWINGS

FIG. 1 shows three different alignments of IFN-alpha sequences:

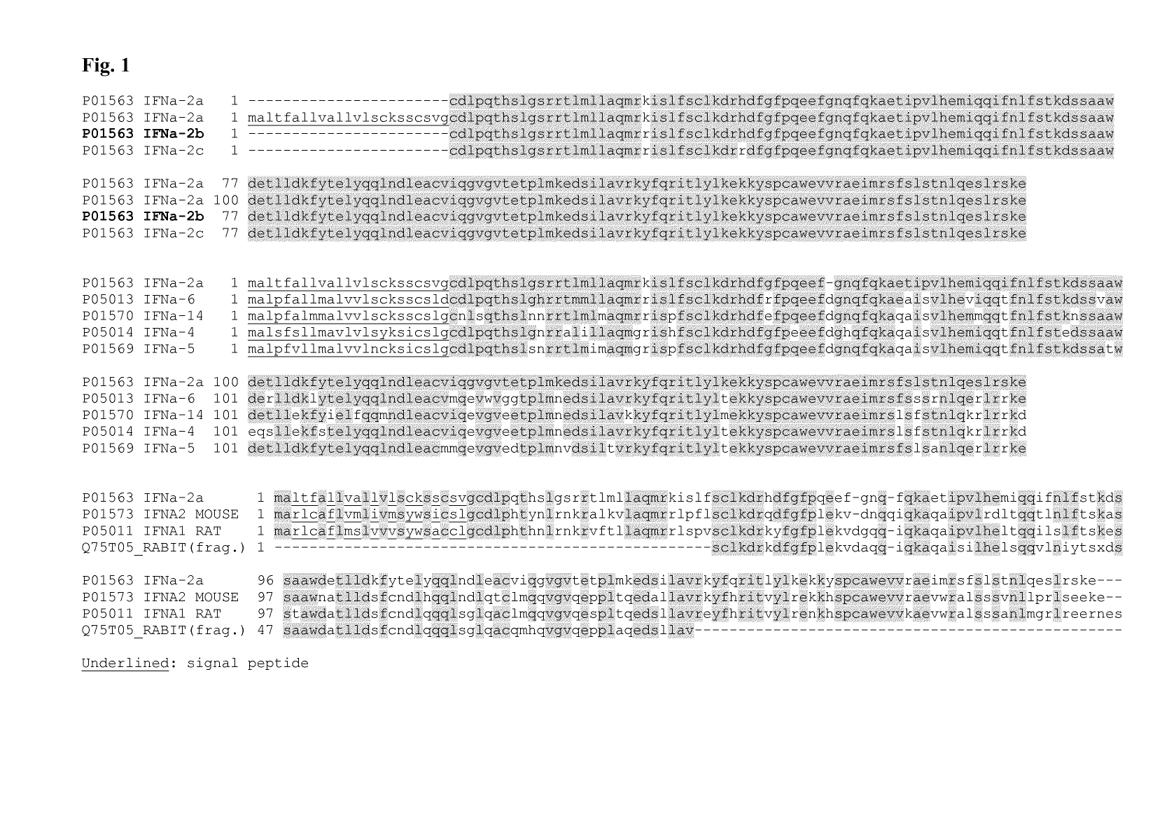

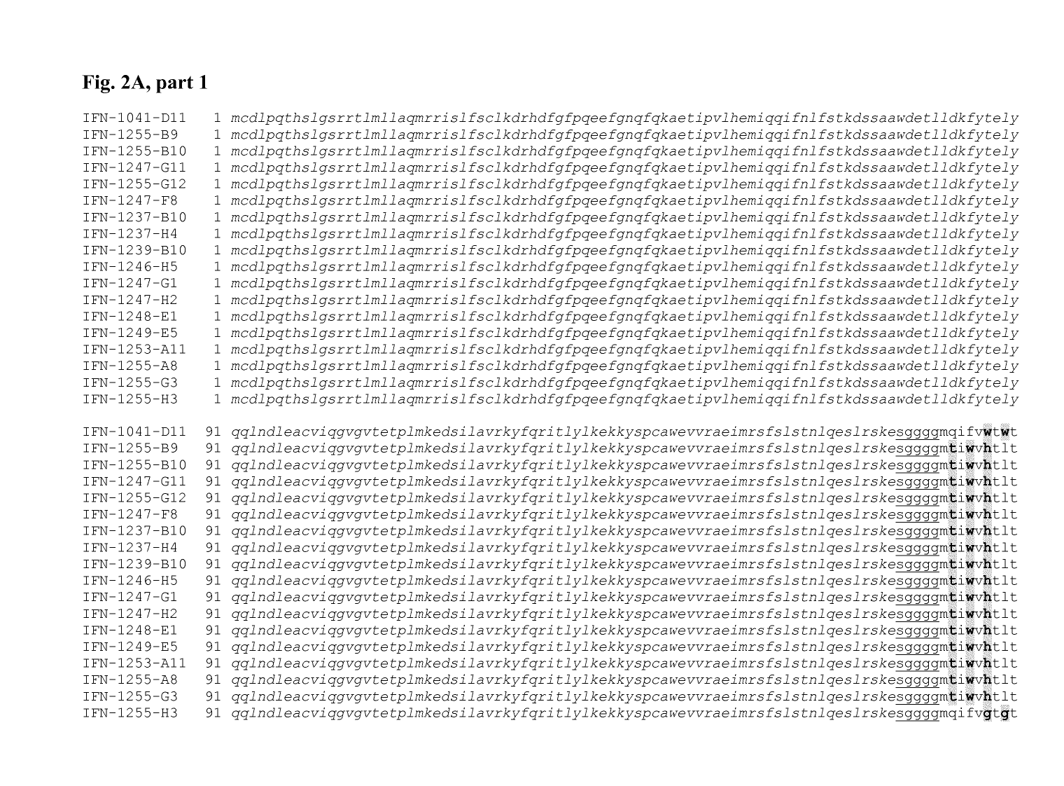

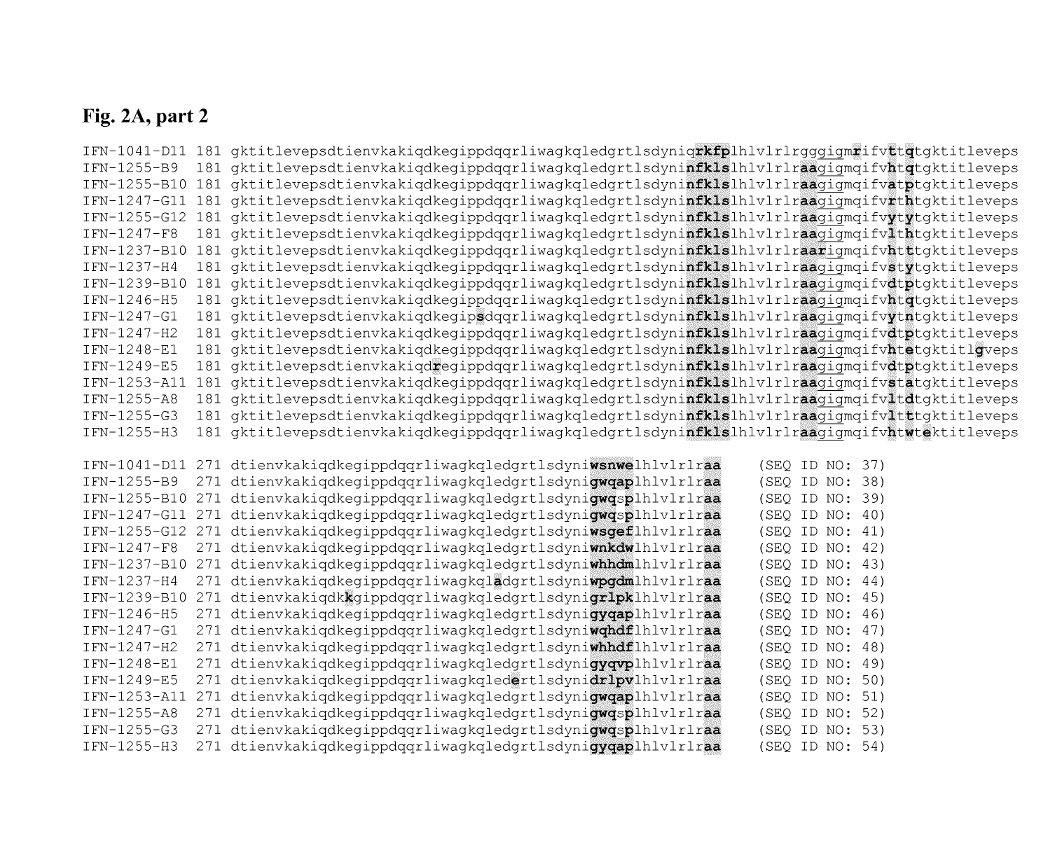

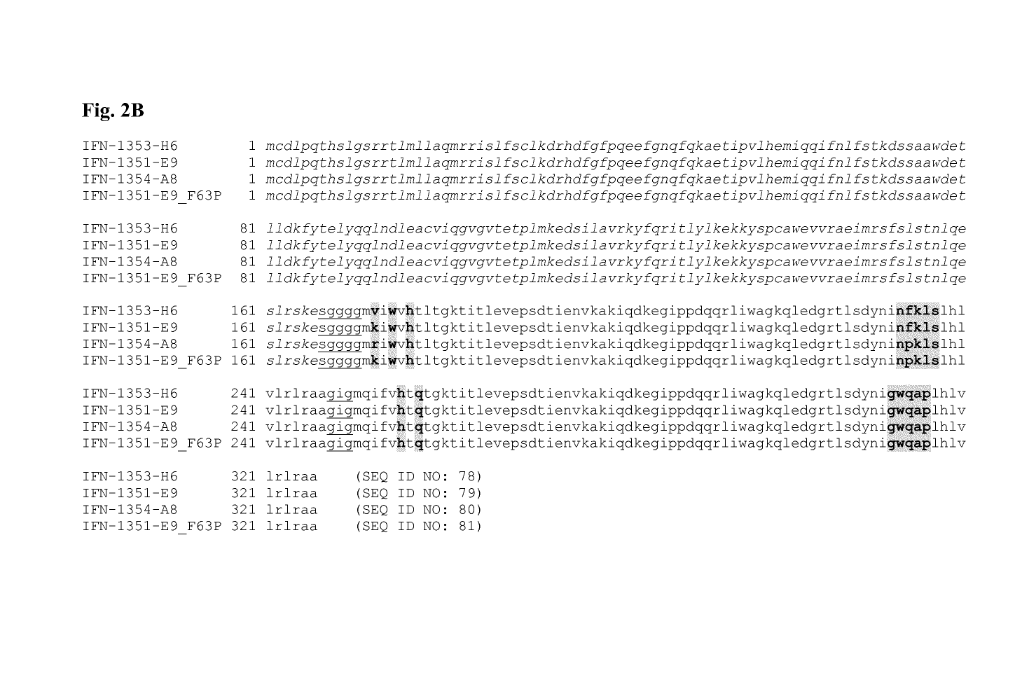

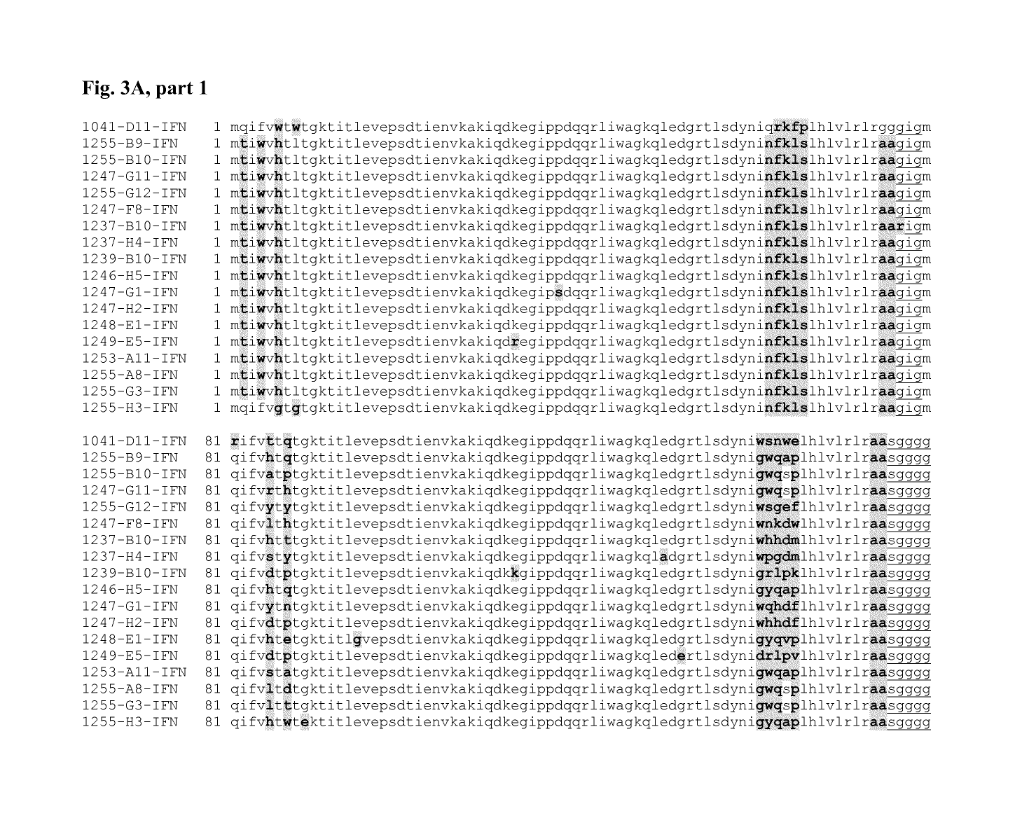

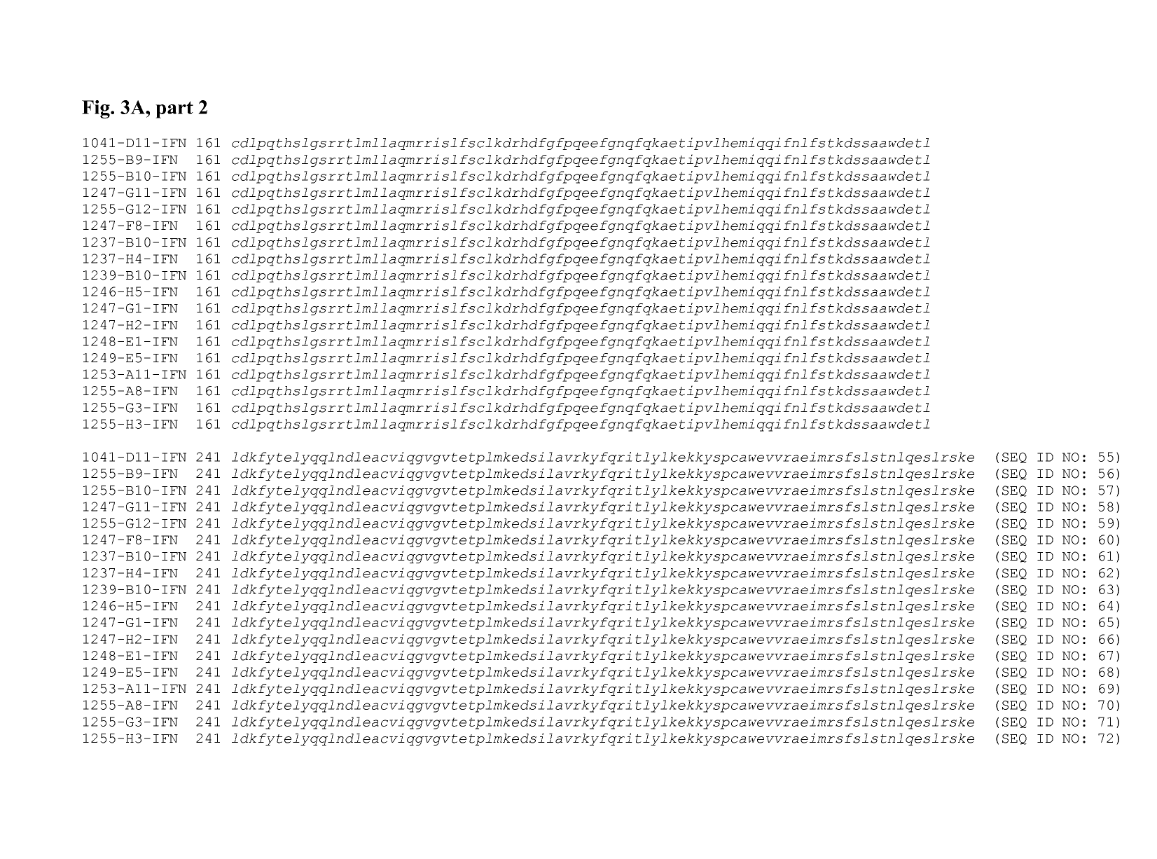

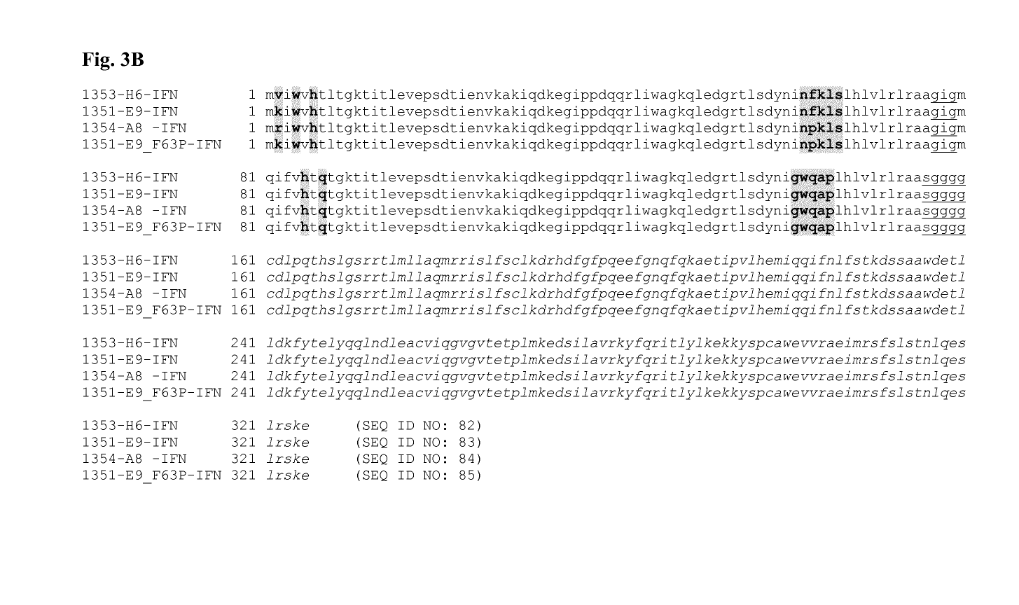

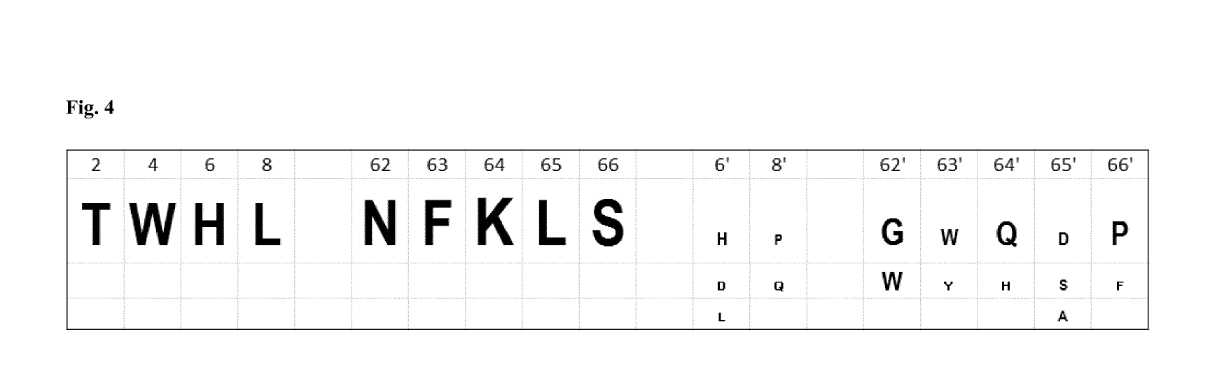

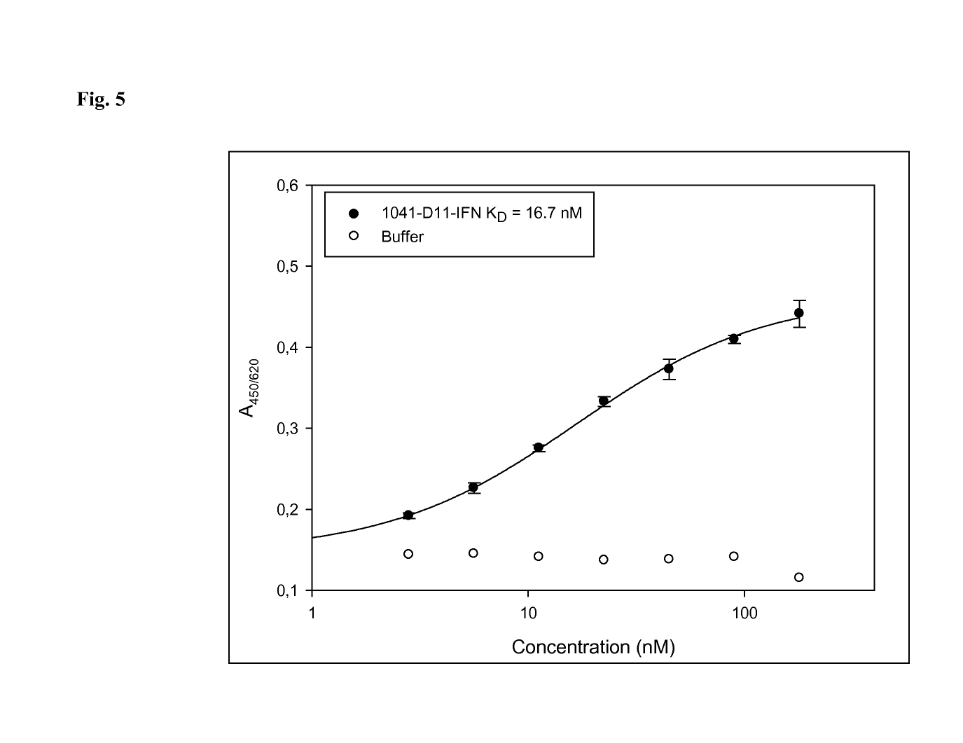

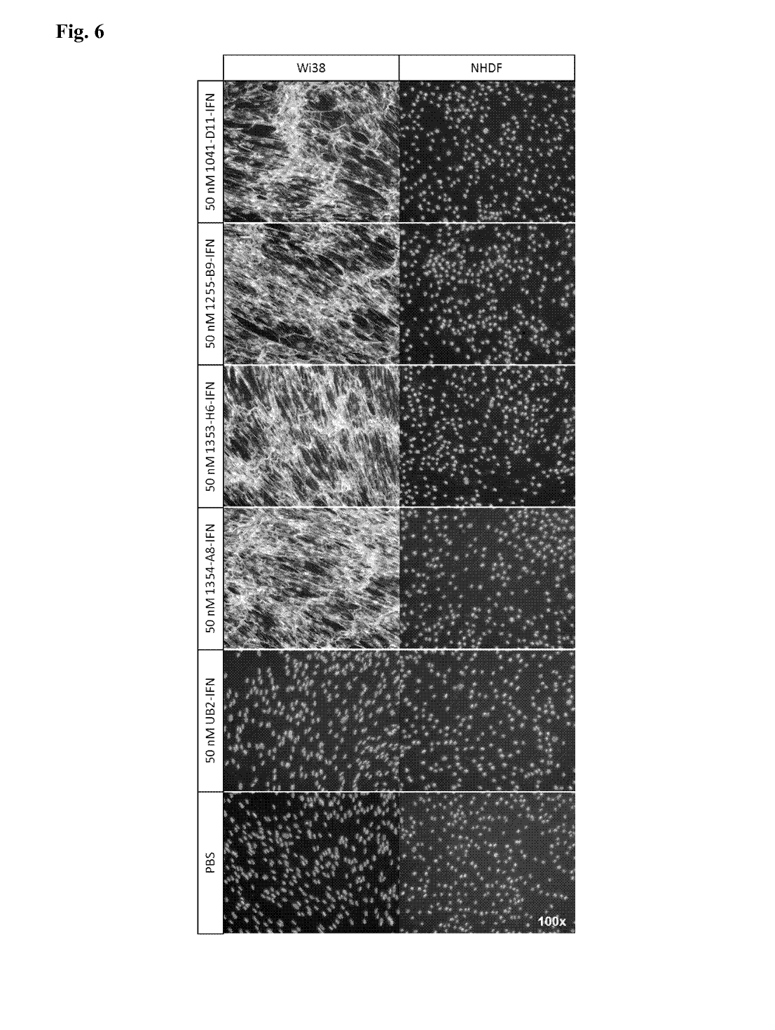

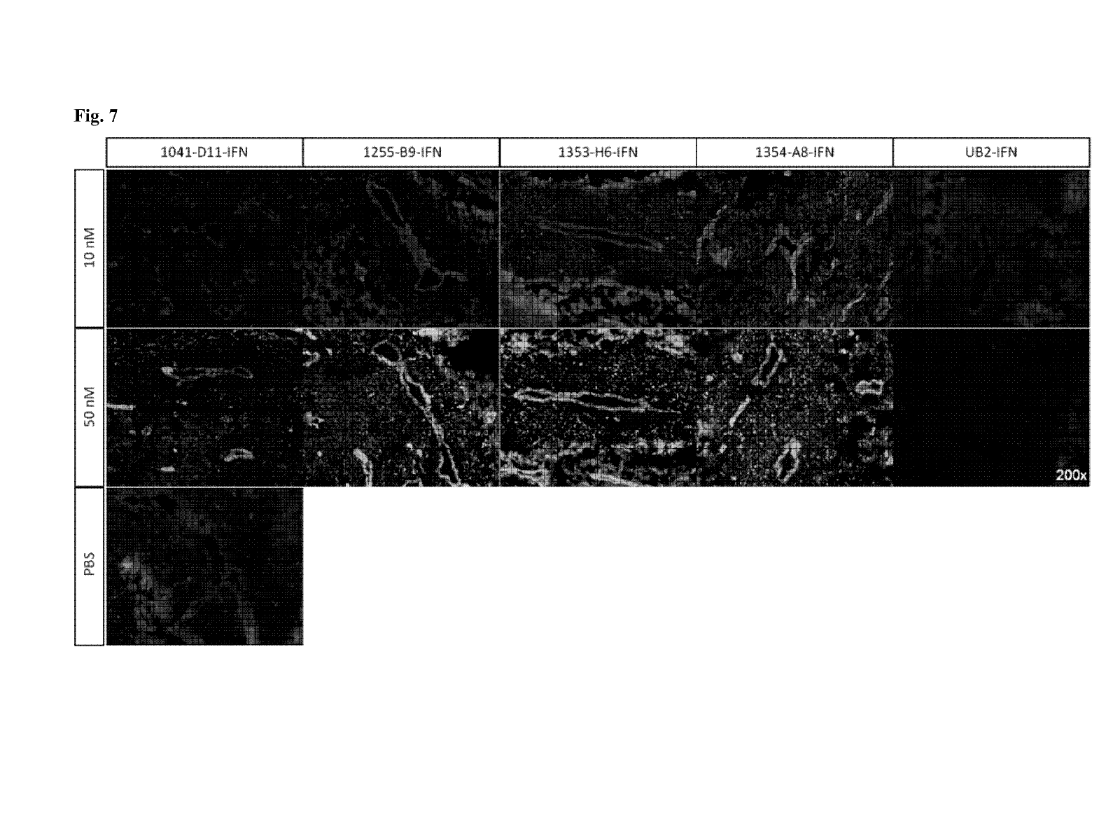

The alignment at the top compares the amino acid sequences of human IFN-alpha-2a without signal peptide (SEQ ID NO: 8), human IFN-alpha-2a with signal peptide (SEQ ID NO: 9), human IFN-alpha-2b without signal peptide (SEQ ID NO: 10), and human IFN-alpha-2c without signal peptide (SEQ ID NO: 11).

The alignment in the middle compares the amino acid sequences of human IFN-alpha-2a (SEQ ID NO: 9), IFN-alpha-6 (SEQ ID NO: 12), IFN-alpha-14 (SEQ ID NO: 13), IFN-alpha-4 (SEQ ID NO: 14), and IFN-alpha-5 (SEQ ID NO: 15). All IFN-alpha forms are shown with their signal peptides.

The alignment at the bottom compares IFN-alpha sequences from different species, namely human IFN-alpha-2a (SEQ ID NO: 9), murine IFN-alpha-2 (SEQ ID NO: 16), rat IFN-alpha-1 (SEQ ID NO: 17), and a fragment of rabbit IFN-alpha (SEQ ID NO: 18).

Grey background highlights amino acids that are identical in human IFN-alpha-2a and the respective IFN-alpha molecule.

FIG. 2 shows alignments of the amino acid sequences of several fusion proteins of the invention.

FIG. 2A shows an alignment of the amino acid sequences of IFN-1041-D11 (SEQ ID NO: 37), IFN-1255-B9 (SEQ ID NO: 38), IFN-1255-B10 (SEQ ID NO: 39), IFN-1247-G11 (SEQ ID NO: 40), IFN-1255-G12 (SEQ ID NO: 41), IFN-1247-F8 (SEQ ID NO: 42), IFN-1237-B10 (SEQ ID NO: 43), IFN-1237-H4 (SEQ ID NO: 44), IFN-1239-B10 (SEQ ID NO: 45), IFN-1246-H5 (SEQ ID NO: 46), IFN-1247-G1 (SEQ ID NO: 47), IFN-1247-H2 (SEQ ID NO: 48), IFN-1248-E1 (SEQ ID NO: 49), IFN-1249-E5 (SEQ ID NO: 50), IFN-1253-A11 (SEQ ID NO: 51), IFN-1255-A8 (SEQ ID NO: 52), IFN-1255-G3 (SEQ ID NO: 53), and IFN-1255-H3 (SEQ ID NO: 54).

FIG. 2B shows an alignment of the amino acid sequences of IFN-1353-H6 (SEQ ID NO: 78), IFN-1351-E9 (SEQ ID NO: 79), IFN-1354-A8 (SEQ ID NO: 80), and IFN-1351-E9_F63P (SEQ ID NO: 81).

The modified monomeric ubiquitin subunits presented in FIG. 2A and FIG. 2B are based on ubiquitin F45W, i.e. an ubiquitin mutein which differs from the wild-type sequence according to SEQ ID NO: 1 by an amino acid exchange F45W.

The interferon alpha 2b sequence is shown in italics in FIG. 2A and FIG. 2B. Substitutions relevant for the target binding are highlighted in the ubiquitin monomers by using bold-type and a grey background. The amino acid exchange F45W is not highlighted. Linker regions are underlined. The exchange in position 75 and 76 (G75A and G76A) is not important for binding ED-B (therefore not highlighted).

FIG. 3 shows alignments of the amino acid sequences of several fusion proteins of the invention.

FIG. 3A shows an alignment of the amino acid sequences of 1041-D11-IFN (SEQ ID NO: 55), 1255-B9-IFN (SEQ ID NO: 56), 1255-B10-IFN (SEQ ID NO: 57), 1247-G11-IFN (SEQ ID NO: 58), 1255-G12-IFN (SEQ ID NO: 59), 1247-F8-IFN (SEQ ID NO: 60), 1237-B10-IFN (SEQ ID NO: 61), 1237-H4-IFN (SEQ ID NO: 62), 1239-B10-IFN (SEQ ID NO: 63), 1246-H5-IFN (SEQ ID NO: 64), 1247-G1-IFN (SEQ ID NO: 65), 1247-H2-IFN (SEQ ID NO: 66), 1248-E1-IFN (SEQ ID NO: 67), 1249-E5-IFN (SEQ ID NO: 68), 1253-A11-IFN (SEQ ID NO: 69), 1255-A8-IFN (SEQ ID NO: 70), 1255-G3-IFN (SEQ ID NO: 71), and 1255-H3-IFN (SEQ ID NO: 72).

FIG. 3B shows an alignment of the amino acid sequences of 1353-H6-IFN (SEQ ID NO: 82), 1351-E9-IFN (SEQ ID NO: 83), 1354-A8-IFN (SEQ ID NO: 84), and 1351-E9_F63P-IFN (SEQ ID NO: 85).

The modified monomeric ubiquitin subunits presented in FIG. 3A and FIG. 3B are based on ubiquitin F45W, i.e. an ubiquitin mutein which differs from the wild-type sequence according to SEQ ID NO: 1 by an amino acid exchange F45W.

The interferon alpha 2b sequence is shown in italics in FIG. 3A and FIG. 3B. Substitutions relevant for the target binding are highlighted in the ubiquitin subunits by using bold-type and a grey background. The amino acid exchange F45W is not highlighted. Linker regions are underlined. The exchange in position 75 and 76 (G75A and G76A) is not important for binding ED-B (therefore not highlighted).

FIG. 4 shows a consensus-sequence of 18 modified hetero-dimeric ubiquitin protein parts present in the fusion proteins shown in FIGS. 2A and 3A. Said 18 modified hetero-dimeric ubiquitin protein parts are shown in the sequence listing under SEQ ID NOs: 19 to 36. FIG. 4 lists only those amino acid positions, which were randomized. Numbers 2, 4, 6, 8, 62, 63, 64, 65, and 66 refer to the amino acid positions in the N-terminal ubiquitin monomer (“first monomer”), while numbers 6′, 8′, 62′, 63′, 64′, 65′, and 66′ refer to the amino acid positions in the C-terminal ubiquitin monomer (“second monomer”).

FIG. 5 shows the functionality of the interferon-domain of the fusion protein of the invention. The binding is shown by closed circles connected by a fitted line. The interferon α/β receptor binds to the fusion protein (1041-D11-IFN; SEQ ID NO: 55) with an affinity of 16.7 nM=1.67×10⁻⁸ M.

FIG. 6 shows the analyses of the different cancer-binding proteins fused to IFN-alpha on different cell types. The first column shows the staining on the ED-B-expressing cell line Wi38. The second column shows the analyses on NHDF cell line as control. 1353-H6-IFN and 1354-H8-IFN show a strong binding with 50 nM on ED-B-containing extracellular matrix from Wi38-cells. The detection of 1041-D11-IFN and 1255-B9-IFN is not as strong as the binding of 1353-H6-IFN and 1354-H8-IFN (row 1). No binding on NHDF-cells can be observed (column 2). The protein UB2-IFN-alpha (fusion protein of two pre-modified, non-targeting ubiquitin monomers (SEQ ID NO: 91) and interferon alpha which has no specific cancer targeting function) IFN-alpha and the PBS control in the row 5 and 6 show no staining.

FIG. 7 shows the binding of different concentrations of 1041-D11-IFN, 1255-B9-IFN, 1353-H6-IFN, 1354-A8-IFN and UB2-IFN on F9-tumor slices. The variants 1255-B9-IFN (column 2), 1353-H6-IFN (column 3) and 1354-A8-IFN (column 4) provide a strong ED-B binding with the concentration of 50 nM. 1041-D11-IFN shows a weaker ED-B-staining (column 1). The non-targeting protein UB2-IFN is not detected on F9-tumor slices. No unspecific staining of the anti-IFN-alpha-antibody was detected (row 3).

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents (for example: patents, patent applications, scientific publications, manufacturer's specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

Sequences: All sequences referred to herein are disclosed in the attached sequence listing that, with its whole content and disclosure, is a part of this specification.

The term “about” when used in connection with a numerical value is meant to encompass numerical values within a range having a lower limit that is 5% smaller than the indicated numerical value and having an upper limit that is 5% larger than the indicated numerical value.

The term “extra-domain B of fibronectin” or briefly designated as “ED-B” comprises all proteins which show a sequence identity to SEQ ID NO: 2 of at least 70%, optionally 75%, further optionally at least 80%, 85%, 90%, 95%, 96% or 97% or most preferably showing a sequence identity to SEQ ID NO: 2 of 100% and having the above defined functionality of ED-B (see in particular the above section entitled “Extra-domain B of fibronectin as tumor specific protein”).

The terms “protein capable of binding” or “binding protein” refer to an ubiquitin protein comprising a binding domain to a target molecule (e.g. a tumor antigen such as ED-B) as further defined below. Any such binding protein based on ubiquitin may comprise additional protein domains that are not binding domains, such as, for example, multimerization moieties, polypeptide tags, polypeptide linkers and/or non-proteinaceous polymer molecules. Some examples of non-proteinaceous polymer molecules are hydroxyethyl starch, polyethylene glycol, polypropylene glycol, or polyoxyalkylene.

Antibodies and fragments thereof are well known to the person skilled in the art. The binding protein of the invention is not an antibody or a fragment thereof, such as Fab or scFv fragments. Further, the binding domain of the invention does not comprise an immunoglobulin fold as present in antibodies.

In the present specification, the terms “ligand” and “target molecule” and “binding partner” are used synonymously and can be exchanged. A ligand is any molecule (e.g. an antigen or a hapten) capable of binding with an affinity as defined herein to the hetero-multimeric modified ubiquitin protein.

Preferred “target molecules” when practicing the present invention are proteins and more specifically antigenic epitopes present on proteins. More preferred “target molecules” are tumor antigens, such as proteins or epitopes that are present on the outside of a tumor cell but that are absent on normal cells of the same tissue-type or which are present in tumor tissue but absent on normal tissue from the same tissue type. A particularly preferred “target molecule” in the context of the present invention is ED-B of fibronectin.

The term “ubiquitin protein” covers the ubiquitin in accordance with SEQ ID NO: 1 and modifications thereof according to the following definition. Ubiquitin is highly conserved in eukaryotic organisms. For example, in all mammals investigated up to now ubiquitin has the identical amino acid sequence. Particularly preferred are ubiquitin molecules from humans, rodents, pigs, and primates. Additionally, ubiquitin from any other eukaryotic source can be used. For instance ubiquitin of yeast differs only in three amino acids from the sequence of SEQ ID NO: 1. Generally, the ubiquitin proteins covered by said term “ubiquitin protein” show an amino acid identity of more than 70%, preferably of more than 75%, preferably of more than 80%, of more than 85%, of more than 90%, of more than 95%, of more than 96% of more than 97%, or of more than 98% to SEQ ID NO: 1. As used herein, the term “ubiquitin protein” also covers ubiquitin-derived proteins that exhibit an amino acid identity of at least 70%, preferably at least 75%, more preferably at least 80%, at least 85%, at least 90%, at least 95%, at least 96%, at least 97%, or at least 98% to SEQ ID NO: 1.

The term “a modified ubiquitin protein” refers to modifications of the ubiquitin protein, any one of substitutions, insertions or deletions of amino acids or a combination thereof, while substitutions are the most preferred modifications which may be supplemented by any one of the modifications described above. The number of modifications is strictly limited as said modified monomeric ubiquitin units have an amino acid identity to SEQ ID NO: 1 of one of the group consisting of at least 80%, at least 83%, at least 85%, at least 87% and at least 90%. At the most, the overall number of substitutions in a monomeric unit is, therefore, limited to 15 amino acids corresponding to 80% amino acid identity. The total number of modified amino acids in the hetero-dimeric ubiquitin molecule is 30 amino acids corresponding to 20% amino acid modifications based on the hetero-dimeric protein. The amino acid identity of the dimeric modified ubiquitin protein compared to a dimeric unmodified ubiquitin protein with a basic monomeric sequence of SEQ ID NO: 1 is selected from one of the group consisting of at least 80%, at least 83%, at least 84%, at least 85%, at least 86%, at least 87% at least 88%, at least 89%, and at least 90%.

For determining the extent of sequence identity between two amino acid sequences, for example, the SIM Local similarity program (Xiaoquin Huang and Webb Miller, Advances in Applied Mathematics, vol. 12: 337-357, 1991) or ClustalW can be used (Thompson et al., Nucleic Acids Res., 22(22): 4673-4680, 1994). In particular, the sequence identity percentage between a derivative of ubiquitin and the amino acid sequence of SEQ ID NO: 1 can be determined with either of these programs. Preferably, the default parameters of the SIM Local similarity program or of ClustalW are used, when calculating sequence identity percentages. Preferably, the extent of the sequence identity of the modified protein to SEQ ID NO: 1 is determined relative to the complete sequence of SEQ ID NO: 1.

In the context of the present invention, the extent of sequence identity between a modified sequence and the sequence from which it is derived (also termed: “parent sequence”) is generally calculated with respect to the total length of the unmodified sequence, if not explicitly stated otherwise.

A “dimer” is considered as a protein in this invention which comprises two monomeric ubiquitin proteins. If the dimer comprises two differently modified monomers, it is called a “heteromeric-dimer” or “hetero-dimer”. Thus, the “hetero-dimer” of the invention is considered as a fusion of two differently modified monomeric ubiquitin proteins exhibiting a combined binding property (binding domain) for its specific target molecule (e.g. a tumor antigen such as ED-B or any other antigens).

It is emphasized that the modified hetero-dimeric ubiquitin protein of the invention is not obtained by separately screening each monomeric ubiquitin protein and combining two of them afterwards but by screening for hetero-dimeric proteins consisting of a first and a second monomeric unit which exhibit together a binding activity to the target molecule. It is to be expected that each of said subunits exhibit a quite limited binding affinity towards the target molecule while only the combined dimeric modified ubiquitin protein will have the excellent binding properties described herein.

After having received the sequence information on the most potent binding ubiquitin molecules, these molecules may be obtained by any other method, e.g. by chemical synthesis or by genetic engineering methods, e.g. by linking the two already identified monomeric ubiquitin units together.

An advantage of multimerization of differently modified ubiquitin monomers in order to generate hetero-multimeric binding proteins (e.g. hetero-dimeric proteins) with binding activity lies in the increase of the total number of amino acid residues that can be modified to generate a new high affinity binding property to a target molecule (e.g. a tumor antigen such as ED-B). The main advantage is that while even more amino acids are modified, the protein-chemical integrity is maintained without decreasing the overall stability of the scaffold of said newly created binding protein to the target molecule (e.g. a tumor antigen such as ED-B). The total number of residues which can be modified in order to generate a novel binding site for the target molecule is increased as the modified residues can be allocated to two monomeric ubiquitin proteins. Thus, the number of modifications permissible in a modified hetero-dimeric protein is twice the number of modifications in a modified monomeric ubiquitin molecule. A modular structure of the ubiquitin-based target molecule binding protein allows increasing the overall number of modified amino acids as said modified amino acids are included on two monomeric ubiquitin molecules. The present method provides for the identification of hetero-dimeric ubiquitin molecules having one specificity for a target molecule (e.g. a tumor antigen such as ED-B).

Thus, the use of hetero-dimers having a common binding site for binding partners opens up the possibility to introduce an increased number of modified residues which do not unduly influence the protein-chemical integrity of the final binding molecule, since the overall amount of those modified residues is distributed over the two monomeric units which form the dimer. Said hetero-dimeric modified ubiquitin proteins binding to a target molecule (e.g. a tumor antigen such as ED-B) are present in a library of proteins.

Both binding regions form a binding site which is formed as a contiguous region of amino acids on the surface of the hetero-dimeric modified ubiquitin protein so that said modified ubiquitin is feasible to bind much more efficient to the target molecule (e.g. a tumor antigen such as ED-B) than each monomeric protein taken alone. According to the present invention the two monomeric proteins are not linked together after having screened the most potent binding ubiquitin molecules but that already the screening process is performed in the presence of the hetero-dimeric ubiquitins. After having received the sequence information on the most potent binding ubiquitin molecules, these molecules may be obtained by any other method, e.g. by chemical synthesis or by genetic engineering methods, e.g. by linking the two already identified monomeric ubiquitin units together.

According to the invention, the two differently modified ubiquitin monomers which bind to one ligand are to be linked by head-to-tail fusion to each other using e.g. genetic methods. The differently modified fused ubiquitin monomers are only effective if both “binding domain regions” act together. A “binding domain region” is defined herein as region on a ubiquitin monomer that has modified amino acids in 1-8 amino acids, preferably at least 3 amino acids, more preferably at least 5 amino acids, and most preferred at least 6 amino acids of regions 2-8 and 62-68, preferably of amino acid positions 2, 4, 6, 8, 62, 63, 64, 65, 66, 68 of SEQ ID NO: 1 or SEQ ID NO: 91 which are involved in binding the target.

A “head to-tail fusion” is to be understood as fusing the C-terminus of the first protein to the N-terminus of the second protein. In a head-to-tail fusion, monomers may be connected directly without any linker, i.e. by a direct peptide bond. Alternatively, the fusion of ubiquitin monomers can be performed via linkers.

As used herein, the term “linker” refers to a molecule that joins at least two other molecules either covalently or noncovalently, e.g., through hydrogen bonds, ionic or van der Waals interactions, e.g., a nucleic acid molecule that hybridizes to one complementary sequence at the 5′ end and to another complementary sequence at the 3′ end, thus joining two non-complementary sequences. A “linker” is to be understood in the context of the present application as a moiety that connects a first polypeptide with at least a further polypeptide. The second polypeptide may be the same as the first polypeptide or it may be different.

Preferred herein are peptide linkers. This means that the peptide linker is an amino acid sequence that connects a first polypeptide with a second polypeptide. The peptide linker is connected to the first polypeptide and to the second polypeptide by a peptide bond. Typically, a peptide linker has a length of between 1 and 20 amino acids; e.g. 1, 2, 3, 4, 5, 6, 7, 8, 9, 10, 11, 12, 13, 14, 15, 16, 17, 18, 19, or 20 amino acids. It is preferred that the amino sequence of the peptide linker is not immunogenic to human beings. For example, a linker having at least the amino acid sequence GIG (SEQ ID NO: 3) or having at least the amino acid sequence SGGGG (SEQ ID NO: 4) or any other linker, for example GIG (SEQ ID NO: 3), RIG (SEQ ID NO: 73), SGGGG (SEQ ID NO: 4), SGGGGIG (SEQ ID NO: 5), SGGGGSGGGGIG (SEQ ID NO: 6), SGGGGSGGGG (SEQ ID NO: 7), SG, (SGGG)ₙ (i.e. n repetitions of SEQ ID NO: 88, wherein n is between 1 and 10 (e.g. n may be 1, 2, 3, 4, 5, 6, 7, 8, 9 or 10)), or (GGGS)ₙ (i.e. n repetitions of SEQ ID NO: 87, wherein n is between 1 and 10 (e.g. n may be 1, 2, 3, 4, 5, 6, 7, 8, 9 or 10)), or any other peptide linker can be used. Also other linkers for the genetic fusion of two ubiquitin monomers are known in the art and can be used.

Likewise, the hetero-dimeric ubiquitin protein may be connected to the interferon directly without any linker in the fusion proteins of the invention. Alternatively, the fusion of the hetero-dimeric ubiquitin protein to the interferon can be performed via linkers, such as the linkers defined by SEQ ID NOs: 3, 4, 5, 6, 7, and 73. Other linkers for the genetic fusion of two proteins are known to the person skilled in the art.

The modified ubiquitin proteins of the invention are engineered, artificial proteins with novel binding affinities to target molecules (e.g. tumor antigens such as ED-B). This means that the binding affinity to a target was created de novo by substituting certain amino acids in wildtype ubiquitin. After substituting 1-8 amino acids in a ubiquitin monomer and linking two modified ubiquitin monomers, these novel artificial protein—heterodimeric ubiquitin—has binding capabilities that did not exist before.

The term “substitution” comprises also the chemical modification of amino acids by e.g. substituting or adding chemical groups or residues to the original amino acid. The substitution of amino acids in at least one surface-exposed region of the protein comprising amino acids located in at least one beta sheet strand of the beta sheet region or positioned up to 3 amino acids adjacent to the beta sheet strand is crucial.

The substitution of amino acids for the generation of the novel binding domain specific to the target molecules (e.g. tumor antigens such as ED-B) can be performed according to the invention with any desired amino acid, i.e. for the modification to generate the novel binding property to the target molecule it is not mandatory to take care that the amino acids have a particular chemical property or a side chain, respectively, which is similar to that of the amino acids substituted so that any amino acid desired can be used for this purpose.

The step of modification of the selected amino acids is performed according to the invention preferably by mutagenesis on the genetic level by random mutagenesis, i.e. a random substitution of the selected amino acids. Preferably, the modification of ubiquitin is carried out by means of methods of genetic engineering for the alteration of a DNA belonging to the respective protein. Preferably, expression of the ubiquitin protein is then carried out in prokaryotic or eukaryotic organisms.

Substitutions are performed particularly in surface-exposed amino acids of the four beta strands of the beta sheets or surface exposed amino acids up to 3 amino acids adjacent to the beta sheet strand of ubiquitin protein. Each beta strand consists usually of 5-7 amino acids. With reference to SEQ ID NO: 1, for example, the beta strands usually cover amino acid residues 2-7, 12-16, 41-45 and 65-71. Regions which may be additionally and preferably modified include positions up to 3 amino acids (i.e. 1, 2, or 3) adjacent to the beta sheet strand. The preferred regions which may be additionally and preferably modified include in particular amino acid residues 8-11, 62-64 and 72-75. The preferred regions include beta turns which link two beta strands together. One preferred beta-turn includes amino acid residues 62-64. A most preferred amino acid which is closely adjacent to the beta sheet strand is the amino acid in position 8. In addition, further preferred examples for amino acid substitutions are positions 36, 44, 70, and/or 71. For example, those regions which may be additionally and preferably modified include amino acids 62, 63, and 64 (3 amino acids), or 72, 73 (2 amino acids), or 8 (1 amino acid).

In preferred embodiments, the amino acid residues are altered by amino acid substitutions. However, also deletions and/or insertions are allowable. The number of amino acids which may be added is limited to 1, 2, 3, 4, 5, 6, 7, or 8 amino acids in a monomeric ubiquitin subunit, and accordingly 1, 2, 3, 4, 5, 6, 7, 8, 9, 10, 11, 12, 13, 14, 15, 16 amino acids with respect to the dimeric ubiquitin protein. The number of amino acids which may be deleted is limited to 1, 2, 3, 4, 5, 6, 7, or 8 amino acids in a monomeric ubiquitin subunit, and accordingly 1, 2, 3, 4, 5, 6, 7, 8, 9, 10, 11, 12, 13, 14, 15, 16 amino acids with respect to the dimeric ubiquitin protein. In one embodiment, no amino acid insertions are made. In a still further embodiment, no deletions have been performed. In still other embodiments, a number of deletion (e.g. 1, 2, 3, 4, 5, 6, 7, 8, 9, 10, 11, 12, 13, 14, 15, 16 deletions in the hetero-dimeric ubiquitin protein) is combined with a number of insertions (e.g. 1, 2, 3, 4, 5, 6, 7, 8, 9, 10, 11, 12, 13, 14, 15, 16 insertions in the hetero-dimeric ubiquitin protein).

Provided that the modified ubiquitin protein of the present invention comprises additionally to said substitutions specified in the claims and explained herein also deletions and/or additions of one or more amino acids, the amino acid positions given for wild type human ubiquitin (SEQ ID NO: 1) have to be aligned with the modified ubiquitin in order to allot the corresponding proteins to each other. In case of fusion proteins (see below), the numbering (and alignment) of each of the monomeric ubiquitin subunits is done in the same way, i.e. an alignment of, for example, a dimer is started at amino acid position 1 for each respective subunit.

In monomeric ubiquitin, preferably from mammals, e.g. human, at least 10% (corresponds to about 2 amino acids) of the amino acids present in beta strands, preferably at least 20% (corresponds to about 4 amino acids), further preferably at least 22% (corresponds to about 5 amino acids), can be modified, preferably substituted, according to the present invention to generate a binding property that did not exist previously. Additionally to modifications in beta strands, further amino acids can be modified, in particular, closely adjacent to beta strands. At a maximum, up to about 30% or up to about 25% are modified, preferably substituted. In one beta strand, generally one to four amino acids are modified. In one embodiment, amino acids of two beta strands are modified. Preferably, three of six amino acids in preferably the first and the fourth beta strand, e.g. region of amino acid residues 2-7 or 65-71, are modified.

The modified amino acids of a modified monomeric ubiquitin according to the invention used as building unit for a hetero-dimer accounts for minimal 6% to in total up to 20% of amino acids (corresponding to about 5 to about 15 amino acids). Considering this, there is a sequence identity to SEQ ID NO: 1 of the modified ubiquitin protein to at least 80%. In further embodiments of the invention, the sequence identity on amino acid level to the amino acid sequence of SEQ ID NO: 1 is at least 83%, at least 85%, at least 87% and furthermore at least 90%, at least 92% or at least 95%. The invention covers also amino acid sequence identities of more than 97% of the modified ubiquitin protein compared to the amino acid sequence of SEQ ID NO: 1.

In a further embodiment of the invention, an ubiquitin is modified in 5 or 6 or 7 amino acids in regions 2-8 and/or 62-68, preferably selected from positions 2, 4, 6, 8, 62, 63, 64, 65, 66, and/or 68 of SEQ ID NO: 1. In another embodiment, the ubiquitin to be modified in these positions, was already pre-modified. For example, further modifications could comprise modifications at amino acids 74 and/or 75 and/or 76 and/or at amino acid 45 to generate better stability or protein-chemical properties but without any influence on the novel binding property. A modified ubiquitin monomer is obtainable wherein in total up to 5, 6, 7, 8, 9, 10, 11, 12, 13, 14 and a maximum of 15 amino acids of the ubiquitin of SEQ ID NO: 1 are modified, preferably substituted. According to an example, a modified monomeric ubiquitin could be obtained having 14 substitutions and a deletion. Based on the total number of amino acids of ubiquitin this corresponds to a percentage of about 20%. This was surprising since usually a much lower percentage is already sufficient to disturb the folding of the protein.

The “beta sheet structure” is defined by being essentially sheet-like and almost completely stretched. In contrast to alpha helices which are formed from an uninterrupted segment of the polypeptide chain, beta sheets can be formed by different regions of the polypeptide chain. In this way, regions spaced further apart in the primary structure can get into close proximity with each other. A beta strand typically has a length of 5-10 amino acids (usually 5-7 residues in ubiquitin) and has an almost completely stretched conformation. The beta strands come so close to each other that hydrogen bonds form between the C-O group of one strand and the NH group of the other strand and vice versa. Beta-sheets can be formed from several strands and have a sheet-like structure wherein the position of the C alpha atoms alternates between above or below the sheet-like plane. The amino acid side chains follow this pattern and, thus, alternatively point towards the top or towards the bottom. Depending on the orientation of the beta strands the sheets are classified into parallel and antiparallel sheets. According to the invention both can be mutated and used for the preparation of the proteins claimed.

For the mutagenesis of the beta strands and the beta-sheet structure, a beta strand or positions up to 3 amino acids adjacent to the beta strand (which is a strand of the beta sheet) are selected in the ubiquitin that are close to the surface. Surface-exposed amino acids can be identified with respect to the available X-ray crystallographic structure. If no crystal structure is available, attempts can be made by means of computer analysis to predict surface-exposed beta sheet regions and the accessibility of individual amino acid positions with respect to the available primary structure or to model the 3d protein structure and to obtain information about potential surface-exposed amino acids in this manner. Further disclosure thereof can be taken e.g. from Vijay-Kumar S, Bugg C. E., Cook W. J, J. Mol. Biol., 1987 Apr. 5; 194(3):531-44.

It is, however, also possible to carry out modifications in the beta sheet or of positions up to 3 amino acids adjacent to the beta strand for which the time-consuming pre-selection of amino acid positions to be mutagenized can be omitted. Those DNA regions encoding the beta sheet structures or up to 3 amino acids adjacent to the beta sheet strand are isolated from their DNA environment, subjected to random mutagenesis and are afterwards re-integrated into the DNA coding for the protein from which they were removed previously. This is followed by a selection process for mutants with the desired binding properties.

In another embodiment of the invention the beta strands or up to 3 amino acids adjacent to the beta strand close to the surface are selected as already explained above and the amino acid positions to be mutagenized within these selected regions are identified. The amino acid positions selected in this way can then be mutagenized on the DNA level either by site-directed mutagenesis, i.e. a codon coding for a specific amino acid is substituted by a codon encoding another previously selected specific amino acid, or this substitution is carried out in the context of a random mutagenesis wherein the amino acid position to be substituted is defined but not the codon encoding the novel, not yet determined amino acid.

“Surface-exposed amino acids” are amino acids that are accessible to the surrounding solvent. If the accessibility of the amino acids in the protein is more than 8% compared to the accessibility of the amino acid in the model tripeptide Gly-X-Gly, the amino acids are called “surface-exposed”. These protein regions or individual amino acid positions, respectively, are also preferred binding sites for potential binding partners for which a selection shall be carried out according to the invention. In addition, reference is made to Caster et al., 1983 Science, 221, 709-713, and Shrake & Rupley, 1973 J. Mol. Biol. 79(2):351-371, which for complete disclosure are incorporated by reference in this application.

Variations of ubiquitin protein scaffold differing by amino acid substitutions in the region of the de novo generated artificial binding site from the parental protein and from each other can be generated by a targeted mutagenesis of the respective sequence segments. In this case, amino acids having certain properties such as polarity, charge, solubility, hydrophobicity or hydrophilicity can be replaced or substituted, respectively, by amino acids with respective other properties. Besides substitutions, the terms “mutagenesis” and “modified” and “replaced” comprise also insertions and/or deletions. On the protein level the modifications can also be carried out by chemical alteration of the amino acid side chains according to methods known to those skilled in the art.

A “randomly modified nucleotide or amino acid sequence” is a nucleotide or amino acid sequence which in a number of positions has been subjected to insertion, deletion or substitution by nucleotides or amino acids, the nature of which cannot be predicted. In many cases the random nucleotides (amino acids) or nucleotide (amino acid) sequences inserted will be “completely random” (e.g. as a consequence of randomized synthesis or PCR-mediated mutagenesis). However, the random sequences can also include sequences which have a common functional feature (e.g. reactivity with a ligand of the expression product) or the random sequences can be random in the sense that the ultimate expression product is of completely random sequence with e.g. an even distribution of the different amino acids.

In accordance with the invention, the term “Kd” (or its alternative spelling “K_{D}”) defines the specific binding affinity which is in accordance with the invention in the range of 10⁻⁷-10⁻¹² M. A value of 10⁻⁵ M and below can be considered as a quantifiable binding affinity. Depending on the application a value of 10⁻⁷ M to 10⁻¹¹ M is preferred for e.g. chromatographic applications or 10⁻⁹ to 10⁻¹² M for e.g. diagnostic or therapeutic applications. Further preferred binding affinities are in the range of 10⁻⁷ to 10⁻¹⁰ M, preferably to 10⁻¹¹ M. The methods for determining the binding affinities are known per se and can be selected for instance from the following methods: ELISA, Surface Plasmon Resonance (SPR) based technology (offered for instance by Biacore®), fluorescence spectroscopy, isothermal titration calorimetry (ITC), analytical ultracentrifugation, FACS.

The term “fusion protein” relates to a fusion protein comprising a binding or non-binding protein of the invention fused to a functional or an effector component. In one embodiment, the invention relates to a fusion protein comprising a hetero-dimeric binding protein of the invention as targeting moiety fused to a functional or an effector domain, such as interferon. A fusion protein of the invention may further comprise non-polypeptide components, e.g. non-peptidic linkers, non-peptidic ligands, e.g. for therapeutically or diagnostically relevant radionuclides. It may also comprise small organic or non-amino acid based compounds, e.g. a sugar, oligo- or polysaccharide, fatty acid, etc. In one preferred embodiment of the invention, the hetero-dimeric ubiquitin-based ED-B binding molecule is covalently or non-covalently conjugated to a protein or peptide having therapeutically relevant properties. Methods for covalently and non-covalently attaching a protein of interest to a support are well known in the art, and are thus not described in further detail here.

The term “biologically active mutein” of an interferon encompasses polypeptides that are sequence variants of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 or SEQ ID NO: 18 and exhibit the same biological functions as the naturally occurring interferon molecules according to SEQ ID NOs: 8 to 18. Such “biologically active mutein” molecules can occur in nature or can be artificially created polypeptides. In the context of the present application, the term “biologically active mutein of interferon” especially refers to polypeptides that exhibit at least 90% sequence identity (e.g. at least 91%, at least 92%, at least 93%, at least 94%, at least 95%, at least 96%, at least 97%, at least 98%, or at least 99%) to the amino acid sequence set forth in any one of SEQ ID NOs: 8 to 18, (particularly to SEQ ID NO: 8 or 10) and that exhibit an activity, as does a naturally occurring interferon molecule with an amino acid sequence as set forth in any one of SEQ ID NOs: 8 to 18 (particularly SEQ ID NO: 8 or 10, respectively). A sequence variant of the amino acid sequences according to SEQ ID NOs: 8 to 18 (preferably SEQ ID NO: 8 or 10) is considered to be a “biologically active mutein of interferon” polypeptide for the purposes of the present invention, if said sequence variant exhibits at least 90% of the activity of human interferon (in particular interferon alpha or interferon beta; more preferably interferon alpha 2a or 2b) having the amino acid sequence according to SEQ ID NO: 8 or 10. The activity can be determined, for example, by a receptor binding assay as explained in example 3 of this application or by an ISRE-reporter gene assay as detailed in example 8 of this application.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

A “composition” according to the present invention comprises at least two pharmacologically active compounds. These compounds can be administered simultaneously or separately with a time gap of one minute to several days. The compounds can be administered via the same route or differently; e.g. oral administration of one active compound and parenteral administration of another are possible. Also, the active compounds may be formulated in one medicament, e.g. in one infusion solution or as a kit comprising both compounds formulated separately. Also, it is possible that both compounds are present in two or more packages.

A “combination preparation” according to the present invention comprises a fusion protein of the invention together with a pharmaceutically active agent, preferably a cytotoxic or cytostatic or anti-cancer agent.

### EMBODIMENTS OF THE INVENTION

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

In a first aspect the present invention is directed to a fusion protein comprising, essentially consisting of or consisting of the following parts:

(i) an interferon or a biologically active mutein thereof;
(ii) a modified hetero-dimeric ubiquitin protein that is capable of binding to a target molecule; and
(iii) optionally a linker.

In preferred embodiments of the first aspect, the interferon is interferon-alpha (IFN-α) or interferon-beta (IFN-β). In particularly preferred embodiments, the interferon is an IFN-α selected from the group consisting of IFN-α 2a, IFN-α 2b, IFN-α 2c, IFN-α 6, IFN-α 14, IFN-α 4, IFN-α 5, and biologically active muteins of any of these. More preferred is IFN-alpha 2, even more preferred IFN-alpha 2b. Since IFN-alpha 2a and IFN-alpha 2b have a particular high affinity to the same receptor, both IFN-alpha molecules could be used as fusion partner for the targeting moiety.

In preferred embodiments of the first aspect, the modified hetero-dimeric ubiquitin protein has a specific binding affinity to the target molecule of Kd≦10⁻⁷, preferably ≦10⁻⁸, more preferably ≦10⁻⁹, even more ≦10⁻¹⁰, and most preferably ≦10⁻¹¹.

In preferred embodiments of the first aspect, the target molecule is a tumor antigen. In particularly preferred embodiments, the target molecule is the extradomain B (ED-B) of fibronectin.

In preferred embodiments of the first aspect, the modified hetero-dimeric ubiquitin protein comprises two monomeric ubiquitin units linked together in a head-to-tail arrangement. In some embodiments, these two monomeric ubiquitin units are directly linked, i.e. without a linker. Alternatively, these two monomeric ubiquitin units may be linked by a linker sequence. Preferably said linker comprises, essentially consists of or consists of an amino acid sequence selected from the following amino acid sequences: GIG (SEQ ID NO: 3), RIG (SEQ ID NO: 73), SGGGG (SEQ ID NO: 4), SGGGGIG (SEQ ID NO: 5), SGGGGSGGGGIG (SEQ ID NO: 6), SGGGGSGGGG (SEQ ID NO: 7), the dipeptide linker SG, (SGGG)ₙ (i.e. n repetitions of SEQ ID NO: 88, wherein n is between 1 and 10 (e.g. n may be 1, 2, 3, 4, 5, 6, 7, 8, 9 or 10)), or (GGGS)ₙ (i.e. n repetitions of SEQ ID NO: 87, wherein n is between 1 and 10 (e.g. n may be 1, 2, 3, 4, 5, 6, 7, 8, 9 or 10)).

Regions to be Modified in Ubiquitin

The regions for modification can be basically selected as to whether they can be accessible for the target molecule as binding partner and whether the overall structure of the protein will presumably show tolerance to a modification. Particularly preferred are two regions for modification of amino acids within the ubiquitin molecule. The first preferred region is between amino acids 2-8, the second region is between amino acids 62-68.

Besides modifications in surface-exposed beta strands also modifications in other surface-exposed regions of the protein can be carried out, preferably in positions up to 3 amino acids adjacent to the beta strand. These modified regions are involved in the newly generated binding with high affinity to ED-B.

In another optional embodiment of the present invention amino acids in one or two, preferably two of the four beta strands in the protein or positions up to 3 amino acids adjacent to preferably two of the four beta strands are modified to generate a novel binding property. Also optional is a modification in three or four of the four beta strands or positions up to 3 amino acids adjacent to three or four of the beta strands for the generation of an ED-B binding.

It is particularly preferred that amino acids in the amino-terminal and carboxy-terminal strand or in positions up to 3 amino acids adjacent to the amino-terminal and carboxy-terminal strand are modified, preferably substituted, to generate a novel binding site to ED-B. In this respect, it is particularly preferred that up to 4 amino acids adjacent to the carboxy-terminal beta sheet strand are modified, preferably substituted, e.g. in positions 62, 63, and 64, and up to 1 amino acid adjacent to the amino-terminal beta sheet strand is modified, preferably substituted e.g. in position 8.

Particularly preferred is a modification, preferably a substitution, in at least three surface-exposed amino acids of three or more of positions 2, 4, 6, 8, 62, 63, 64, 65, 66, and 68 of a mammalian ubiquitin, preferably human ubiquitin. This is important for the generation of modified proteins having a binding affinity that did not exist previously with respect to the target molecule as binding partner.

In preferred embodiments of the first aspect, each monomeric ubiquitin unit in said modified hetero-dimeric ubiquitin protein is modified independently from the modifications in the other monomeric ubiquitin unit by substitutions of 1-8 amino acids, preferably at least 3 amino acids, more preferably at least 5 amino acids, and most preferred at least 6 amino acids selected from regions 2-8 and/or 62-68, preferably selected from positions 2, 4, 6, 8, 62, 63, 64, 65, 66, and 68 of SEQ ID NO: 1 or SEQ ID NO: 91. Optionally 1, 2, 3, 4, 5, 6, 7, 8, 9 or 10 of said amino acid residues are modified, optionally in combination with additional amino acid residues. Other positions might be suitable for substitution as well. It is important that by modification of ubiquitin monomers a high specific binding to a cancer target is generated and that the structure of ubiquitin is maintained.

In preferred embodiments of the first aspect, each modified monomeric ubiquitin unit has an amino acid sequence identity of at least 80% (e.g. at least 83%, at least 85%, at least 87%, at least 90%, at least 92%, at least 95%, or at least 97%) to the amino acid sequence defined by SEQ ID NO: 1 or SEQ ID NO: 91.

In preferred embodiments of the first aspect, the substitutions in the monomeric ubiquitin units comprise substitutions in amino acid region 2-8 and in amino acids in region 62-66. In particular, both first monomeric units, substitutions at least in one or more amino acid positions 6, 62, 63, 64, 65, 66 and optionally further modifications, preferably substitutions of other amino acids, are preferred.

Thus, in one embodiment the fusion protein is a genetically fused hetero-dimer of said ubiquitin monomers having different amino acids substitutions in the first ubiquitin monomer and in the second ubiquitin monomer, preferably as shown in Table 1.

TABLE 1
Preferred substitutions in ubiquitin monomers relevant for specific targeting
to a tumor (SEQ ID NO: 19, 20, 21, 74, 75, 76, and 77, respectively)

| | Ubiquitin-Monomer 1 | | | | | | | | | Ubiquitin-Monomer 2 | | | | | | | | |
|---|---|---|---|---|---|---|---|---|---|---|---|---|---|---|---|---|---|---|
| Variant | 2 | 4 | 6 | 8 | 62 | 63 | 64 | 65 | 66 | 2 | 4 | 6 | 8 | 62 | 63 | 64 | 65 | 66 |
| 1041-D11 | | | W | W | | R | K | F | P | R | | T | Q | W | S | N | W | E |
| 1255-B9 | T | W | H | | N | F | K | L | S | | | H | Q | G | W | Q | A | P |
| 1353-H6 | V | W | H | | N | F | K | L | S | | | H | Q | G | W | Q | A | P |
| 1351-E9 | K | W | H | | N | F | K | L | S | | | H | Q | G | W | Q | A | P |
| 1354-A8 | R | W | H | | N | P | K | L | S | | | H | Q | G | W | Q | A | P |
| 1351-E9_F | K | W | H | | N | P | K | L | S | | | H | Q | G | W | Q | A | P |
| Preferred | | | | | | | K | | | | | | Q | | | | | |

In preferred embodiments of the first aspect, the substitutions in the monomeric ubiquitin units comprise

(1) in the first monomeric unit: substitutions at least in one or more positions selected from the group of amino acid positions 6, 8, 63, 64, 65, and 66; and
in the second monomeric unit: substitutions at least in one or more positions selected from the group of amino acid positions 6, 8, 62, 63, 64, 65, and 66; optionally additionally 2, or
(2) in the first monomeric unit: substitutions at least in one or more positions selected from the group of amino acid positions 2, 4, 6, 62, 63, 64, 65, and 66; and
in the second monomeric unit: substitutions at least in one or more positions selected from the group of amino acid positions 6, 8, 62, 63, 64, and 66; optionally additionally 65,
and optionally further modifications, preferably substitutions of other amino acids.

Preferred amino acid substitutions in one or more positions selected from the group of amino acid positions 2, 4, 6, 62, 63, 64, 65, and 66 of the first monomeric unit can be identified from a consensus sequence as shown in FIG. 4. (The leucine residue shown in position 8 is the amino acid present in the wild-type sequence.) Furthermore, preferred amino acid substitutions in one or more positions selected from the group of amino acid positions 6, 8, 62, 63, 64, 65, and 66 of the second monomeric unit can be identified from the consensus sequence shown in FIG. 4. It is most preferred that amino acid position 64 of the first monomer is substituted by a Lysin (K) and that amino acid position 8 of the second monomer is substituted by a Glutamine (Q).

Thus, in one embodiment the fusion protein is a genetically fused hetero-dimer of said ubiquitin monomers having amino acids substitutions in one or more positions selected from the group of amino acid positions 2, 4, 6, and 62-66 of the first ubiquitin monomer and substitutions in amino acid residues in one or more positions selected from the group of amino acid positions 6, 8, 62-64 and 66, and optionally in position 65 of the second ubiquitin monomer, preferably
in the first ubiquitin monomer substitutions
   Glutamine (Q) to threonine (T) in position 2 (or no substitution),
   Phenylalanine (F) to Tryptophan (W) in position 4 (or no substitution),
   Lysine (K) to Histidine (H) in position 6,
   Glutamine (Q) to Asparagine (N) in position 62,
   Lysine (K) to Phenylalanine (F) in Position 63,
   Glutamic acid (E) to Lysine (K) in position 64,
   Serine (S) to Leucine (L) in position 65, and
   Threonine (T) to Serine (S) in position 66;
in the second ubiquitin monomer, the substitutions
   Lysine (K) to Histidine (H), Aspartic acid (D) or Leucine (L) in position 6,
   Leucine (L) to Glutamine (Q) or Proline (P) in position 8,
   Glutamine (Q) to Glycine (G) or Tryptophan (W) in position 62,
   Lysine (K) to an amino acid with an aromatic side chain, preferably
   Tryptophan (W) or Tyrosine (Y), in position 63,
   Glutamic acid (E) to an amino acid containing an —NH₂ group or an —NH— group in the side chain, preferably Glutamine (Q) or Histidine (H), in position 64,
   optionally Serine (S) to an amino acid that has no voluminous side chain,
   preferably Alanine (A) or Aspartic acid (D) in position 65, and
   Threonine (T) to Proline (P) or Phenylalanine (F) in position 66, preferably to
   Proline (P), when the amino acid substitutions Q62G and E64Q are present.

The alternative substitutions in the second monomer can be combined with each other without any limitations provided that the resulting modified ubiquitin hetero-dimers show a specific binding affinity to said extradomain B (ED-B) of fibronectin of Kd≦10⁻⁷ M and provided that the structural stability of the ubiquitin protein is not destroyed or hampered. Other amino acid substitutions could be possible, provided that a specific binding affinity to ED-B of Kd≦10⁻⁷ M is achieved.

In preferred embodiments of the first aspect, from 1 to 7 amino acids are additionally modified in the modified hetero-dimeric ubiquitin protein. Preferably, said from 1 to 7 additionally modified amino acids are selected from one or more of the amino acids in positions 2, 4, 8, 33, 36, 38, 62, 44, 70, and 71 of the first monomeric ubiquitin unit and in positions 2, 10, 16, 34, 36, 44, 51, 53, 65, 70, and 71 of the second monomeric ubiquitin unit.

In some embodiments of the first aspect, additional amino acids are substituted at positions 45, 75 and/or 76 of the first monomeric ubiquitin unit and/or at positions 45, 75 and/or 76 of the second monomeric ubiquitin unit. Preferred substitutions in these positions are one or more substitutions selected from the group consisting of F45W, G75A and G76A.

In particularly preferred embodiments of the present invention, a pre-modified ubiquitin contains all three of the amino acid exchanges F45W, G74A and G75A. Said pre-modified ubiquitin is shown in SEQ ID NO: 91 and is particularly well-suited for practicing the present invention. More specifically, all embodiments of the present invention that refer to the wild-type ubiquitin sequence according to SEQ ID NO: 1 apply in an analogous manner to the amino acid sequence of SEQ ID NO: 91.

Fusion Proteins of the Invention

The invention provides for fusion proteins comprising an interferon or biologically active mutein and a modified hetero-dimeric ubiquitin protein capable of binding to a target molecule. Preferred are cancer targets.

In some embodiments of the first aspect, the linker is absent and the IFN, preferably IFN-α, most preferred IFN-alpha 2, even more preferred IFN-alpha 2a or IFN-alpha 2b, or the biologically active mutein(s) thereof and the modified hetero-dimeric ubiquitin protein are directly fused to each other.

In some embodiments of the first aspect, the IFN, preferably IFN-α, or the biologically active mutein thereof is positioned C-terminally to the modified hetero-dimeric ubiquitin protein. Alternatively, the IFN, preferably IFN-α, or the biologically active mutein thereof is positioned N-terminally to the modified hetero-dimeric ubiquitin protein.

In some other embodiments of the first aspect, the linker is present and the IFN, preferably IFN-α, or the biologically active mutein thereof and the modified hetero-dimeric ubiquitin protein are connected via the linker. In some embodiments, the order of the parts of the fusion protein from the N-terminus to the C-terminus is as follows: modified hetero-dimeric ubiquitin protein-linker-IFN, preferably IFN-α, or biologically active mutein thereof. Alternatively, the order of the parts of the fusion protein from the N-terminus to the C-terminus is as follows: IFN, preferably IFN-α, or biologically active mutein thereof -linker-modified hetero-dimeric ubiquitin protein. Preferably said linker comprises, essentially consists of or consists of an amino acid sequence selected from the following amino acid sequences: GIG (SEQ ID NO: 3), RIG (SEQ ID NO: 73), SGGGG (SEQ ID NO: 4), SGGGGIG (SEQ ID NO: 5), SGGGGSGGGGIG (SEQ ID NO: 6), SGGGGSGGGG (SEQ ID NO: 7), SG, (SGGG)ₙ (i.e. n repetitions of SGGG (SEQ ID NO: 88), wherein n is between 1 and 10 (e.g. n may be 1, 2, 3, 4, 5, 6, 7, 8, 9 or 10)), or (GGGS)ₙ (i.e. n repetitions of GGGS (SEQ ID NO: 87), wherein n is between 1 and 10 (e.g. n may be 1, 2, 3, 4, 5, 6, 7, 8, 9 or 10)).

In further preferred embodiments of the first aspect, the modified hetero-dimeric ubiquitin protein comprises, essentially consists of or consists of an amino acid sequence selected from the group consisting of:

SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 74, SEQ ID NO: 75, SEQ ID NO: 76, SEQ ID NO: 77, and an amino acid sequence that exhibits at least 90% sequence identity to one or more of the amino acid sequences according to SEQ ID NOs: 19 to 36 or 74 to 77.

In further preferred embodiments of the first aspect, the fusion protein comprises, essentially consists of or consists of an amino acid sequence selected from the group consisting of:

SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, and an amino acid sequence that exhibits at least 90% sequence identity to one or more of the amino acid sequences according to SEQ ID NOs: 37 to 72 and/or SEQ ID NOs: 78 to 85.

In some embodiments of the first aspect, a fusion protein of the invention may comprise non-polypeptide components, e.g. non-peptidic linkers, non-peptidic ligands, e.g. for therapeutically relevant radionuclides. It may also comprise small organic or non-amino acid based compounds, e.g. a sugar, oligo- or polysaccharide, fatty acid, etc.

The following gives some examples on how to obtain fusion proteins comprising an interferon part and a hetero-dimeric ubiquitin part:
a) conjugation of the interferon via Lysine residues present in ubiquitin;
b) conjugation of the heterodimeric ubiquitin-based binding protein via Cysteine residues—can be located C-terminally, or at any other position (e.g. amino acid residue 24 or 57); conjugation with maleimid selectable components;
c) peptidic or proteinogenic conjugations—genetic fusions (preferred C- or N-terminal);
d) “Tag”-based fusions—A protein or a peptide located either at the C- or N-terminus of the target protein ED-B. Fusion “tags”, for example poly-ionic (poly E/poly R)

These and other methods for covalently and non-covalently attaching a protein of interest to a support are well known in the art, and are thus not described in further detail here.

In a further embodiment of the invention the fusion protein according to the invention may contain artificial amino acids.

A further embodiment relates to fusion proteins according to the invention, further comprising functional components selected from proteins, peptides, polymers (e.g. polyethylene glycol), low molecular weight compounds, sugars and others, as described in WO 2006/040129, which is incorporated herein by reference.

A further embodiment relates to fusion proteins according to the invention, further comprising a component modulating serum half-life, preferably a component selected from the group consisting of polyethylene glycol, albumin-binding peptides, and immunoglobulin.

In a second aspect the present invention is directed to the fusion protein according to the first aspect for use in medicine.

In a third aspect the present invention is directed to the fusion protein according to the first aspect for use in the treatment of cancer or infectious diseases.

The third aspect of the present invention can alternatively be worded as follows: In a third aspect the present invention is directed to a method for treating cancer or infectious diseases, comprising the step: administering a therapeutic amount of the fusion protein according to the first aspect to a subject in need thereof.

In preferred embodiments of the third aspect, the cancer is selected from the group consisting of melanoma, renal cell cancer, hairy cell leukemia, chronic myelogenous leukaemia, multiple myeloma, follicular lymphoma, cutaneous T cell lymphoma, carcinoid tumour, glioblastoma multiforme (brain), breast cancer, lung cancer, adenocarcinoma of the lungs, colorectal cancer, mesothelioma, squamous cell carcinoma, liver cancer, small cell carcinoma, large cell carcinoma, non-small cell lung cancer, pancreas, and Hodgkin lymphoma.

In preferred embodiments of the third aspect, the infectious diseases are selected from the group consisting of long-term hepatitis B and long-term hepatitis C.

In a fourth aspect the present invention is directed to a polynucleotide encoding the fusion protein as defined in the first aspect. In a further embodiment of the fourth aspect, the polynucleotide is for use in medicine, e.g. for use in the treatment of cancer or infectious diseases.

One embodiment of the present invention pertains to a method for treating cancer or infectious diseases, comprising the step: administering a therapeutic amount of the polynucleotide according to the fourth aspect to a subject in need thereof. The cancer to be treated in accordance with the fourth aspect is preferably selected from the same list of cancers as defined above for the third aspect. Likewise, the infectious diseases to be treated in accordance with the fourth aspect are preferably selected from the same list of infectious diseases as defined above for the third aspect.

In some embodiments of the fourth aspect, polynucleotides are operatively linked to expression control sequences allowing expression of the fusion proteins of the invention in prokaryotic and/or eukaryotic host cells. Such expression control sequences include but are not limited to inducible and non-inducible, constitutive, cell cycle regulated, metabolically regulated promoters, enhancers, operators, silencers, repressors and other elements that are known to those skilled in the art and that drive or otherwise regulate gene expression. Such regulatory elements include but are not limited to regulatory elements directing constitutive expression like, for example, promoters transcribed by RNA polymerase III like, e.g. promoters for the snRNA U6 or scRNA 7SK gene, the cytomegalovirus hCMV immediate early gene, the early or late promoters of SV40 adenovirus, viral promoter and activator sequences derived from, e.g. NBV, HCV, HSV, HPV, EBV, HTLV, MMTV or HIV; which allow inducible expression like, for example, CUP-I promoter, the tet-repressor as employed, for example, in the tet-on or tet-off systems, the lac system, the trp, system; regulatory elements directing tissue specific expression, regulatory elements directing cell cycle specific expression like, for example, cdc2, cdc25C or cyclin A; or the TAC system, the TRC system, the major operator and promoter regions of phage A, the control regions of fd coat protein, the promoter for 3-phosphoglycerate kinase, the promoters of acid phosphatase, and the promoters of the yeast α- or a-mating factors.

In a fifth aspect the present invention is directed to a vector comprising the polynucleotide of the fourth aspect. In a further embodiment of the fifth aspect, the vector is for use in medicine, e.g. for use in the treatment of cancer or infectious diseases.

One embodiment of the present invention pertains to a method for treating cancer or infectious diseases, comprising the step: administering a therapeutic amount of the vector according to the fifth aspect to a subject in need thereof. The cancer to be treated in accordance with the fifth aspect is preferably selected from the same list of cancers as defined above for the third aspect. Likewise, the infectious diseases to be treated in accordance with the fifth aspect are preferably selected from the same list of infectious diseases as defined above for the third aspect.

Vectors suitable for use in the present invention comprises without limitation plasmids, phagemids, phages, cosmids, artificial mammalian chromosomes, knock-out or knock-in constructs, viruses, in particular adenoviruses, vaccinia viruses, attenuated vaccinia viruses, canary pox viruses, lentivirus, herpes viruses, in particular Herpes simplex virus (HSV-I), baculovirus, retrovirus, adeno-associated-virus (AAV), rhinovirus, human immune deficiency virus (HIV), filovirus and engineered versions thereof, virosomes, “naked” DNA liposomes, and nucleic acid coated particles, in particular gold spheres.

In order to express cDNAs encoding the receptors, one typically subclones receptor cDNA into an expression vector that contains a strong promoter to direct transcription, a transcription/translation terminator, and a ribosome-binding site for translational initiation. Suitable bacterial promoters are well known in the art, e.g., E. coli, Bacillus sp., and Salmonella, and kits for such expression systems are commercially available. Similarly eukaryotic expression systems for mammalian cells, yeast, and insect cells are well known in the art and are also commercially available. The eukaryotic expression vector may be, for example an adenoviral vector, an adeno-associated vector, or a retroviral vector.

In a sixth aspect the present invention is directed to a host cell comprising: a fusion protein as defined in the first aspect; a polynucleotide as defined in the fourth aspect; or a vector as defined in the fifth aspect. In a further embodiment of the sixth aspect, the host cell is for use in medicine, e.g. for use in the treatment of cancer or infectious diseases.

One embodiment of the present invention pertains to a method for treating cancer or infectious diseases, comprising the step: administering a therapeutic amount of the host cell according to the sixth aspect to a subject in need thereof. The cancer to be treated in accordance with the sixth aspect is preferably selected from the same list of cancers as defined above for the third aspect. Likewise, the infectious diseases to be treated in accordance with the sixth aspect are preferably selected from the same list of infectious diseases as defined above for the third aspect.

A host cell according to the sixth aspect includes but is not limited to prokaryotic cells such as bacteria (for example, E. coli or B. subtilis), which can be transformed with, for example, recombinant bacteriophage DNA, plasmid DNA, or cosmid DNA expression vectors containing the polynucleotide molecules of the invention; simple eukaryotic cells like yeast (for example, Saccharomyces and Pichia), which can be transformed with, for example, recombinant yeast expression vectors containing the polynucleotide molecule of the invention; insect cell systems like, for example, Sf9 or Hi5 cells, which can be infected with, for example, recombinant virus expression vectors (for example, baculovirus) containing the polynucleotide molecules; amphibian cells, e.g. Xenopus oocytes, which can be injected with, for example, plasmids; plant cell systems, which can be infected with, for example, recombinant virus expression vectors (for example, cauliflower mosaic virus (CaMV) or tobacco mosaic virus (TMV) or transformed with recombinant plasmid expression vectors (for example, Ti plasmid) containing polynucleotide sequences of the invention; or mammalian cell systems (for example, COS, CHO, BHK, HEK293, VERO, HeLa, MDCK, Wi38, and NIH 3T3 cells), which can be transformed with recombinant expression constructs containing, for example, promoters derived, for example, from the genome of mammalian cells (for example, the metallothionein promoter) from mammalian viruses (for example, the adenovirus late promoter and the vaccinia virus 7.5K promoter) or from bacterial cells (for example, the tet-repressor binding is employed in the tet-on and tet-off systems). Also useful as host cells are primary or secondary cells obtained directly from a mammal and transfected with a plasmid vector or infected with a viral vector. Depending on the host cell and the respective vector used to introduce the polynucleotide of the invention the polynucleotide can integrate, for example, into the chromosome or the mitochondrial DNA or can be maintained extrachromosomally like, for example, episomally or can be only transiently comprised in the cells.

In a seventh aspect the present invention is directed to a pharmaceutical composition comprising

a fusion protein as defined in the first aspect;
a polynucleotide as defined in the fourth aspect;
a vector as defined in the fifth aspect; or
a host cell as defined in the sixth aspect
and further comprising a pharmaceutically acceptable carrier.

Fusion proteins according to the invention may be prepared by any of the many conventional and well known techniques such as plain organic synthetic strategies, solid phase-assisted synthesis techniques or by commercially available automated synthesizers. On the other hand, they may also be prepared by conventional recombinant techniques alone or in combination with conventional synthetic techniques.

In an eighth aspect the present invention is directed to a method for generation of a fusion protein as defined in the first aspect, said method comprising the following steps:

(a) providing a population of differently modified dimeric ubiquitin proteins originating from monomeric ubiquitin proteins, said population comprising dimeric ubiquitin proteins comprising two modified ubiquitin monomers linked together, preferably in a head-to-tail arrangement, wherein each monomer of said dimeric protein is differently modified by substitutions of at least 1-8 amino acids, preferably at least 3 amino acids, more preferably at least 5 amino acids, and most preferred at least 6 amino acids in regions 2-8 and/or 62-68, preferably positions 2, 4, 6, 8, 62, 63, 64, 65, 66 and 68 of SEQ ID NO: 1 or SEQ ID NO: 91,

(b) providing a target molecule as potential ligand;

(c) contacting said population of differently modified proteins with said target molecule;

(d) identifying a modified dimeric ubiquitin protein by a screening process, wherein said modified dimeric ubiquitin protein binds to said target molecule with a specific binding affinity of Kd≦10⁻⁷ M (preferably ≦10⁻⁸, more preferably ≦10⁻⁹, even more ≦10⁻¹⁰, and most preferably ≦10⁻¹¹);

(e) isolating said modified dimeric ubiquitin protein with said binding affinity; and

(f) fusing IFN, preferably IFN-α, or a biologically active mutein thereof to the modified dimeric ubiquitin protein obtained in step e).

In preferred embodiments of the eighth aspect, said substitutions comprise
(1) in the first monomeric unit substitutions at least in amino acid positions 6, 8, 63, 64, 65, and 66; and in the second monomeric unit substitutions at least in amino acid positions 6, 8, 62, 63, 64, 65, and 66; optionally additionally 2; or

(2) in the first monomeric unit substitutions at least in amino acid positions 2, 4, 6, 62, 63, 64, 65, and 66; and in the second monomeric unit substitutions at least in amino acid positions 6, 8, 62, 63, 64, and 66; optionally additionally 65.

In preferred embodiments of the eighth aspect, the target molecule is a tumor antigen. In particularly preferred embodiments, the target molecule is the extradomain B (ED-B) of fibronectin.

Optionally, the modification may be performed by genetic engineering on the DNA level and expression of the modified protein in prokaryotic or eukaryotic organisms or in vitro. In a further embodiment, the modification includes a chemical synthesis step.

In one embodiment, said population of differently modified proteins is obtained by genetically fusing two DNA libraries encoding each for differently modified monomeric ubiquitin proteins.

In another embodiment, a modified protein can further be prepared by chemical synthesis. In this embodiment the steps c) to d) of this aspect are then performed in one step.

In an ninth aspect the present invention is directed to a method for the preparation of a fusion protein as defined in the first aspect, said method comprising the following steps:
(a) preparing a nucleic acid encoding a fusion protein as defined in the first aspect;
(b) introducing said nucleic acid into an expression vector;
(c) introducing said expression vector into a host cell;
(d) cultivating the host cell;
(e) subjecting the host cell to culturing conditions under which a fusion protein is expressed from said vector, thereby producing a fusion protein as defined in the first aspect; and
(f) optionally enriching or isolating the fusion protein produced in step (e).

In one embodiment of the ninth aspect, the fusion protein produced in step (e) is in the form of inclusion bodies.

In preferred embodiment of the ninth aspect, the method further comprising the steps: isolating the inclusion bodies; solubilizing said inclusion bodies, thereby obtaining soluble fusion proteins; and further purifying the soluble fusion proteins obtained in the preceding step by at least two chromatographic steps. Suitable chromatographic steps include without limitation hydrophobic interaction chromatography, size-exclusion chromatography, anion exchange chromatography and cation exchange chromatography.

### Methods of Mutagenesis of Ubiquitin

By way of example, the cDNA of ubiquitin, which can be prepared, altered, and amplified by methods known to those skilled in the art, can be used as a starting point for the mutagenesis of the respective sequence segments. For site-specific alteration of ubiquitin in relatively small regions of the primary sequence (about 1-3 amino acids) commercially available reagents and methods are on hand (“Quick Change”, Stratagene; “Mutagene Phagemid in vitro Mutagenesis Kit”, Bio-Rad). For the site-directed mutagenesis of larger regions specific embodiments of e.g. the polymerase chain reaction (PCR) are available to those skilled in the art. For this purpose a mixture of synthetic oligodeoxynucleotides having degenerated base pair compositions at the desired positions can be used for example for the introduction of the mutation. This can also be achieved by using base pair analogs which do not naturally occur in genomic DNA, such as e.g. inosine.

Starting point for the mutagenesis of one or more beta strands of the beta sheet region or positions up to 3 amino acids adjacent to the beta sheet strand can be for example the cDNA of ubiquitin or also the genomic DNA. Furthermore, the gene coding for the ubiquitin protein can also be prepared synthetically.

Different procedures known per se are available for mutagenesis, such as methods for site-specific mutagenesis, methods for random mutagenesis, mutagenesis using PCR or similar methods.

In a preferred embodiment of the invention the amino acid positions to be mutagenized are predetermined. The selection of amino acids to be modified is carried out to meet the predetermined limitations with respect to those amino acids which have to be modified. In each case, a library of different mutants is generally established which is screened using methods known per se. Generally, a pre-selection of the amino acids to be modified can be particularly easily performed as sufficient structural information is available for the ubiquitin protein to be modified.

Methods for targeted mutagenesis as well as mutagenesis of longer sequence segments, for example by means of PCR, by chemical mutagenesis or using bacterial mutator strains also belong to the prior art and can be used according to the invention.

In one embodiment of the invention the mutagenesis is carried out by assembly of DNA oligonucleotides carrying the amino acid codon NNK. It should be understood, however, that also other codons (triplets) can be used. The mutations are performed in a way that the beta sheet structure is preferably maintained. Generally, the mutagenesis takes place on the outside of a stable beta sheet region exposed on the surface of the protein. It comprises both site-specific and random mutagenesis. Site-specific mutagenesis comprising a relatively small region in the primary structure (about 3-5 amino acids) can be generated with the commercially available kits of Stratagene® (QuickChange®) or Bio-Rad® (Mutagene® phagemid in vitro mutagenesis kit) (cf. U.S. Pat. No. 5,789,166; U.S. Pat. No. 4,873,192).

If more extended regions are subjected to site-specific mutagenesis a DNA cassette must be prepared wherein the region to be mutagenized is obtained by the assembly of oligonucleotides containing the mutated and the unchanged positions (Nord et al., 1997 Nat. Biotechnol. 8, 772-777; McConell and Hoess, 1995 J. Mol. Biol. 250, 460-470.). Random mutagenesis can be introduced by propagation of the DNA in mutator strains or by PCR amplification (error-prone PCR) (e.g. Pannekoek et al., 1993 Gene 128, 135 140). For this purpose, a polymerase with an increased error rate is used. To enhance the degree of the mutagenesis introduced or to combine different mutations, respectively, the mutations in the PCR fragments can be combined by means of DNA shuffling (Stemmer, 1994 Nature 370, 389-391). A review of these mutagenesis strategies with respect to enzymes is provided in the review of Kuchner and Arnold (1997) TIBTECH 15, 523-530. To carry out this random mutagenesis in a selected DNA region also a DNA cassette must be constructed which is used for mutagenesis.

Random modification is performed by methods well-established and well-known in the art. In order to introduce the randomized fragments properly into the vectors, it is according to the invention preferred that the random nucleotides are introduced into the expression vector by the principle of site-directed PCR-mediated mutagenesis. However, other options are known to the skilled person, and it is e.g. possible to insert synthetic random sequence libraries into the vectors as well.

To generate mutants or libraries by fusion PCR, for example three PCR reactions may be carried out. Two PCR reactions are performed to generate partially overlapping intermediate fragments. A third PCR reaction is carried out to fuse the intermediate fragments.

The method for construction the library or mutant variants may include constructing a first set of primers around a desired restriction site (restriction site primer), a forward and reverse restriction primer and a second set of primers around, e.g., upstream and downstream of the codon of interest (the mutagenic primers), a forward and reverse mutagenic primer. In one embodiment, the primers are constructed immediately upstream and downstream respectively of the codon of interest. The restriction and mutagenic primers are used to construct the first intermediate and second intermediate fragments. Two PCR reactions produce these linear intermediate fragments. Each of these linear intermediate fragments comprises at least one mutated codon of interest, a flanking nucleotide sequence and a digestion site. The third PCR reaction uses the two intermediate fragments and the forward and reverse restriction primers to produce a fused linear product. The opposite, heretofore unattached ends of the linear product are digested with a restriction enzyme to create cohesive ends on the linear product. The cohesive ends of the linear product are fused by use of a DNA ligase to produce a circular product, e.g. a circular polynucleotide sequence.

To construct the intermediate fragments, the design and synthesis of two sets of forward and reverse primers are performed, a first set containing a restriction enzymes digestion site together with its flanking nucleotide sequence, and the second set contains at least one variant codon of interest (mutagenic primers). Those skilled in the art will recognize that the number of variants will depend upon the number of variant amino acid modifications desired. It is contemplated by the inventor that if other restriction enzymes are used in the process, the exact location of this digestion site and the corresponding sequence of the forward and reverse primers may be altered accordingly. Other methods are available in the art and may be used instead.

It is often necessary to couple the random sequence to a fusion partner by having the randomized nucleotide sequence fused to a nucleotide sequence encoding at least one fusion partner. Such a fusion partner can e.g. facilitate expression and/or purification/isolation and/or further stabilization of the expression product or may involve other favorable effects.

Random substitution of amino acids according to one example of the present invention of 1-8 amino acids, preferably at least 3 amino acids, more preferably at least 5 amino acids and most preferred at least 6 amino acids at regions 2-8 and/or 62-68, preferably positions 2, 4, 6, 8, 62, 63, 64, 65, 66, and/or 68 of monomeric ubiquitin can be performed particularly easily by means of PCR since the positions mentioned are localized close to the amino or the carboxy terminus of the protein. Accordingly, the codons to be manipulated are at the 5′ and 3′ end of the corresponding cDNA strand. Thus, the first oligodeoxynucleotide used for a mutagenic PCR reaction apart from the codons at region 2-8, preferably positions 2, 4, 6, and/or 8 to be mutated—corresponds in sequence to the coding strand for the amino terminus of ubiquitin. Accordingly, the second oligodeoxynucleotide—apart from the codons at region 62-68, preferably of positions 62, 63, 64, 65, 66, and/or 68 to be mutated—at least partially corresponds to the non-coding strand of the polypeptide sequence of the carboxy terminus. By means of both oligodeoxynucleotides a polymerase chain reaction can be performed using the DNA sequence encoding the monomeric ubiquitin as a template.

Furthermore, the amplification product obtained can be added to another polymerase chain reaction using flanking oligodeoxynucleotides which introduce for example recognition sequences for restriction endonucleases. It is preferred according to the invention to introduce the gene cassette obtained into a vector system suitable for use in the subsequent selection procedure for the isolation of ubiquitin variations having binding properties to a predetermined hapten or antigen.

Selection of the Modified Ubiquitin Proteins with Binding Affinity with Respect to the Target Molecule (e.g. ED-B) and Determination of the Modified Amino Acids Responsible for the Binding Affinity

After e.g. at least two different DNA libraries encoding for hetero-dimeric modified ubiquitin proteins have been established by differently modifying selected amino acids in each of the monomeric ubiquitin units, these libraries are genetically fused by e.g. linker technology to obtain DNA molecules encoding for hetero-dimeric modified ubiquitin proteins. The DNA of these libraries is expressed into proteins and the modified dimeric proteins obtained thereby are contacted according to the invention with the target molecule (e.g. a tumor antigen such as ED-B) to optionally enable binding of the partners to each other if a binding affinity does exist.

It is a crucial aspect of the invention that the contacting and screening process is performed already with respect to the hetero-dimeric ubiquitin protein. This process enables screening on those ubiquitin proteins which provide a binding activity to its target molecule. See, for example, Scil Proteins' patent applications WO 2011/073214, WO 2011/073208, and WO 2011/073209 for more details of the selection method; the references are incorporated herein by reference.

Contacting according to the invention is preferably performed by means of a suitable presentation and selection method such as the phage display, ribosomal display, mRNA display or cell surface display, yeast surface display or bacterial surface display methods, preferably by means of the phage display method. For complete disclosure, reference is made also to the following references: Hoess, Curr. Opin. Struct. Biol. 3 (1993), 572-579; Wells and Lowmann, Curr. Opin. Struct. Biol. 2 (1992), 597-604; Kay et al., Phage Display of Peptides and Proteins—A Laboratory Manual (1996), Academic Press. The methods mentioned above are known to those skilled in the art and can be used according to the invention including modifications thereof.

The determination whether the modified protein has a quantifiable binding affinity with respect to a predetermined binding partner can be performed according to the invention preferably by one or more of the following methods: ELISA, plasmon surface resonance spectroscopy, fluorescence spectroscopy, FACS, isothermal titration calorimetry and analytical ultracentrifugation.

### Phage Display Selection Method

One type of phage display procedure adapted to this application is described in the following as an example for a selection procedure according to the invention with respect to variations of ubiquitin which show binding properties. In the same manner e.g. methods for the presentation on bacteria (bacterial surface display; Daugherty et al., 1998, Protein Eng. 11(9):825-832) or yeast cells (yeast surface display; Kieke et al., 1997 Protein Eng. 10(11):1303-10) or cell-free selection systems such as the ribosome display (Hanes and Plückthun, 1997 Proc Natl Acad Sci USA. 94(10):4937-4942; He and Taussig, 1997 Nucleic Acids Res. 25(24):5132-5134) or the cis display (Odegrip et al., 2004 Proc Natl Acad Sci USA. 101(9):2806-2810) or the mRNA display can be applied. In the latter case a transient physical linkage of genotype and phenotype is achieved by coupling of the protein variation to the appropriate mRNA via the ribosome.

In the phage display procedure described herein recombinant variations of ubiquitin are presented on a filamentous phage while the coding DNA of the presented variation is present at the same time packed in a single-stranded form in the phage envelope. Thus, in the frame of an affinity enrichment variations having certain properties can be selected from a library and their genetic information can be amplified by infection of suitable bacteria or added to another cycle of enrichment, respectively. Presentation of the mutated ubiquitin on the phage surface is achieved by genetic fusion to an amino-terminal signal sequence-preferably the PelB signal sequence- and a capsid or surface protein of the phage-preferred is the carboxyterminal fusion to the capsid protein pIII or a fragment thereof. Furthermore, the encoded fusion protein can contain further functional elements such as e.g. an affinity tag or an antibody epitope for detection and/or purification by affinity chromatography or a protease recognition sequence for specific cleavage of the fusion protein in the course of the affinity enrichment. Furthermore, an amber stop codon can be present for example between the gene for the ubiquitin variation and the coding region of the phage capsid protein or the fragment thereof which is not recognized during translation in a suitable suppressor strain partially due to the introduction of one amino acid.

The bacterial vector suitable for the selection procedure in the context of the isolation of ubiquitin variations with binding properties to a target molecule (e.g. ED-B) and into which the gene cassette for the fusion protein described is inserted is referred to as phagemid. Among others, it contains the intergenic region of a filamentous phage (e.g. M13 or f1) or a portion thereof which in the case of a superinfection of the bacterial cell carrying the phagemid by means of helper phages such as e.g. M13K07 results in the packaging of a closed strand of phagemid DNA into a phage capsid. The phagemids generated in this manner are secreted by the bacterium and present the respective ubiquitin variation encoded-due to its fusion to the capsid protein pIII or the fragment thereof-on their surface. Native pIII capsid proteins are present in the phagemid so that its ability to re-infect suitable bacterial strains and therefore the possibility to amplify the corresponding DNA is retained. Thus, the physical linkage between the phenotype of the ubiquitin variation—i.e. its potential binding property—and its genotype is ensured.

Phagemids obtained can be selected with respect to the binding of the ubiquitin variation presented thereon to a target molecule (e.g. ED-B) by means of methods known to those skilled in the art. For this purpose, the presented ubiquitin variations can be transiently immobilized to target substance bound e.g. on microtiter plates and can be specifically eluted after non-binding variations have been separated. The elution is preferably performed by basic solutions such as e.g. 100 mM triethylamine. Alternatively, the elution can be performed under acidic conditions, by proteolysis or direct addition of infected bacteria. The phagemids obtained in this manner can be re-amplified and enriched by successive cycles of selection and amplification of ubiquitin variations with binding properties to a target molecule (e.g. ED-B).

Further characterization of the ubiquitin variations obtained in this way can be performed in the form of the phagemid, i.e. fused to the phage, or after cloning of the corresponding gene cassette into a suitable expression vector in the form of a soluble protein. The appropriate methods are known to those skilled in the art or described in the literature. The characterization can comprise e.g. the determination of the DNA sequence and thus of the primary sequence of the variations isolated. Furthermore, the affinity and specificity of the variations isolated can be detected e.g. by means of biochemical standard methods such as ELISA or plasmon surface resonance spectroscopy, fluorescence spectroscopy, FACS, isothermal titration calorimetry, analytical ultracentrifugation or others. In view of the stability analysis, for example spectroscopic methods in connection with chemical or physical unfolding are known to those skilled in the art.

### Ribosomal Display Selection Method

In a further embodiment of the invention ribosomal display procedure variations of ubiquitin are prepared by means of a cell-free transcription/translation system and presented as a complex with the corresponding mRNA as well as the ribosome. For this purpose, a DNA library as described above is used as a basis in which the genes of variations are present in form of fusions with the corresponding regulatory sequences for expression and protein biosynthesis. Due to the deletion of the stop codon at the 3′ end of the gene library as well as suitable experimental conditions (low temperature, high Mg²⁺ concentration) the ternary complex consisting of the nascent protein, the mRNA and the ribosome is maintained during in vitro transcription/translation.

After a protein library containing hetero-dimeric modified ubiquitin proteins has been established by differently modifying of selected amino acids in each of the monomeric ubiquitin units, the modified dimeric proteins are contacted according to the invention with the ED-B to enable binding of the partners to each other if a binding affinity does exist. These protein libraries may be in the form of a display method library displaying or using any other method presenting the modified proteins in a manner enabling the contact between the modified proteins and the target protein, wherein said display method is optionally a phage display, ribosomal display, TAT phage display, yeast display, bacterial display or mRNA display method.

Selection of the modified ubiquitin variations with respect to their binding activities to their target molecule with a specific binding affinity of Kd in a range of 10⁻⁷-10⁻¹² M can be performed by means of methods known to those skilled in the art. For this purpose, the ubiquitin variations presented e.g. on the ribosomal complexes can be transiently immobilized to target substance bound e.g. on microtiter plates or can be bound to magnetic particles after binding in solution, respectively. Following separation of non-binding variations the genetic information of variations with binding activity can be specifically eluted in the form of the mRNA by destruction of the ribosomal complex. The elution is preferably carried out with 50 mM EDTA. The mRNA obtained in this manner can be isolated and reverse transcribed into DNA using suitable methods (reverse transcriptase reaction), and the DNA obtained in this manner can be re-amplified.

By means of successive cycles of in vitro transcription/translation, selection, and amplification ubiquitin variations with binding properties for a predetermined hapten or antigen can be enriched.

Uses of Preferred Fusion Proteins of the Invention, e.g. Hetero-Dimeric Ubiquitin Based Binding Proteins Specific for ED-B Fused to an Effector Such as Interferon

Since interferons, in particular interferon alpha and interferon beta, show anti-proliferative, anti-viral, and immunomoduling effects, fusion proteins with interferon, preferably interferon alpha or interferon beta, can be used for specific therapies. A fusion protein comprising an interferon as anti-proliferative part and a tumor-specific targeting domain can be directed specifically to the disease site (tumor) and act at the site, thereby reducing side effects which would occur by giving only interferon without a targeting domain. Further, the fusion proteins of the invention, which comprise a modified ubiquitin heterodimer specific for ED-B and an interferon, are to be used for instance for preparing therapeutic means. The fusion proteins according to the invention can be used e.g. as direct effector molecules. Examples of tumors with abundant appearance of ED-B antigen are shown in the Table 2.

TABLE 2
Occurrence of ED-B in Tumors

| Cancer | References (selected examples) |
|---|---|
| Renal cell | Johannsen et al. 2010, Eur J Cancer 46(16): 2926-2935 |
| Melanoma | Frey et al. 2011 Exp Dermatol 20(8): 685-8. |
| Lymphoma | Schliemann et al. 2009, Leuk Res 33(12): 1718-1722 |
| Breast | Midulla et al. 2000 Cancer Res 60(1): 164-169 |
| Colorectal | Midulla et al. 2000 Cancer Res 60(1): 164-169 |
| Head and Neck | Birchler et al. 2003 Laryngoscope 113(7): 1231-1237 |
| Hepatocellular | Menrad and Menssen 2005 |
| | Expert Opin Ther Targets 9(3): 491-500 |
| Lung | Pedretti 2009 Lung Cancer. 64(1): 28-33 |
| Osteosarcoma | Kilian et al. 2004 Bone 35(6): 1334-1345. |
| Pancreas | Wagner et al. 2008 Clin Cancer Res 14(15): 4951-4960 |
| Prostate | Berndt et al. 2010 Histochem Cell Biol 133(4): 467-75 |

Depending on the selected fusion partner the pharmaceutical composition of the invention is adapted to be directed to the treatment of cancer or any other tumor diseases in which ED-B is abundant, such as the tumours listed in Table 2. The most preferred indications for a use of the fusion proteins of the invention are renal cell cancer, melanoma, and lymphoma, but any other indication could be treated.

The compositions are adapted to contain a therapeutically effective dose. The quantity of the dose to be administered depends on the organism to be treated, the type of disease, the age and weight of the patient and further factors known per se.

The compositions contain a pharmaceutically acceptable carrier and optionally can contain further auxiliary agents and excipients known per se. These include for example but not limited to stabilizing agents, surface-active agents, salts, buffers, coloring agents etc.

The pharmaceutical composition can be in the form of a liquid preparation, a cream, a lotion for topical application, an aerosol, in the form of powders, granules, tablets, suppositories, or capsules, in the form of an emulsion or a liposomal preparation. In particular, a combination of different compositions can be used, for example applying the fusion protein of the invention in the form of a liquid preparation, a cream, a lotion for topical application, an aerosol, in the form of powders, granules, tablets, suppositories, or capsules, in the form of an emulsion or a liposomal preparation and the cancer therapeutics as liposomal preparation. The compositions are preferably sterile, non-pyrogenic and isotonic and contain the pharmaceutically conventional and acceptable additives known per se. Additionally, reference is made to the regulations of the U.S. Pharmacopoeia or Remington's Pharmaceutical Sciences, Mac Publishing Company (1990).

In the field of human and veterinary medical therapy and prophylaxis pharmaceutically effective medicaments containing at least one ED-B binding hetero-dimeric ubiquitin protein modified in accordance with the invention can be prepared by methods known per se. Depending on the galenic preparation these compositions can be administered parenterally by injection or infusion, systemically, rectally, intraperitoneally, intramuscularly, subcutaneously, transdermally or by other conventionally employed methods of application. The type of pharmaceutical preparation depends on the type of disease to be treated, the severity of the disease, the patient to be treated and other factors known to those skilled in the art of medicine.

In an embodiment, the pharmaceutical composition contains a protein or a fusion protein of the invention or a combination thereof. In another embodiment, the pharmaceutical composition contains a protein or a fusion protein of the invention or a combination thereof and further comprises one or more cancer therapeutic agents. The fusion protein of the invention can be used in combination with a cytotoxic drug. In an embodiment, the pharmaceutical composition contains a fusion protein of the invention or a combination of two or more fusion proteins of the invention and further comprises one or more cancer therapeutics.

In a preferred embodiment, the cancer therapeutic agent is selected from, for example, but not limited to, the substance classes of alkylating agents, anti-metabolites, mitosis inhibitors and topoisomerase inhibitors, cytotoxic antibiotics, antibiotics, and others. Preferably, cancer therapeutics are selected from the group consisting of CHOP (a combination of cyclophosphamide, vincristine, doxorubicin, and prednisolon), vinblastin, cytarabin, bevacizumab, tumor vaccines, or radiopharmaceuticals or nanoparticular formulations of cytostatics (e.g. Doxil and Abraxane) and others and adjuvants.

Particularly preferred are combinations of the fusion protein of the invention with bevacizumab, CHOP, and vinblastin. Most preferred are combinations of a fusion protein of the invention with bevacizumab.

Further, interferons with extended half-life could be used for the fusion with modified ubiquitins targeted against specific tumor proteins. Several techniques for producing interferons with extended half-life are known in the art.

Fusion proteins with interferon and a modified ubiquitin with binding capabilities for specific viral proteins can be used for the treatment of viral diseases, in particular Hepatitis B, Hepatitis C or Aids (HIV). In embodiments in which the pharmaceutical composition is formulated for the treatment of hepatitis, in particular hepatitis B-virus (HBV) or hepatitis C-virus (HCV)-infections the pharmaceutical composition may additionally comprise other medicaments, preferably Ribavirin. Interferons with extended half-life could be used for the fusion with modified ubiquitins targeted against specific viral proteins. Several techniques for producing interferons with extended half-life are known in the art.

It surprisingly turned out that a fusion protein of a ubiquitin hetero-dimer fused to interferon, wherein the fusion protein preferably has a sequence selected from the group consisting of SEQ ID NOs: 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 78, 79, 80, 81, 82, 83, 84, and 85, can be advantageously applied in therapy. By applying the interferon fusion proteins of the present invention, it is possible to administer interferon in a non-toxic, but still therapeutically effective concentration. Preclinical trials showed superior tumor targeting effect and desired biodistribution. Further, the current invention shows that the IFN-alpha activity is active in the fusion protein. Since interferon is coupled to the tumor targeting fusion protein of the present invention, it can be directly active at the disease site (for example, tumor site) and, thus, the amount of “free” interferon can be drastically reduced (and thus, reducing side effects such as leucopenie, thrombopenie, liver insufficiency, autoimmune diseases, depression). Further, interferon is known for anti-tumoral effects. Thus, by fusion of tumor-specific domain such as the modified ubiquitin proteins of the invention, the interferon can be directed to the disease site. Interferon alpha and Interferon beta are particular preferred because both proteins bind to the same receptor and have comparable effects.

The systemic side effects of interferon can be remarkably reduced by administering interferon as a fusion protein according to the present invention. By using an interferon fusion protein of the invention, the overall dosage of interferon to reach a therapeutic effect thus can be reduced to a large extent and can be advantageously used for systemic tumor treatment in particular in combination with other cancer therapeutics.

In a further embodiment, the pharmaceutical composition is in the form of a kit of parts, providing separated entities for a fusion protein of the invention. In another embodiment, the pharmaceutical composition is in the form of a kit of parts, providing separated entities for a fusion protein of the invention combined with one or more cancer therapeutic agents.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees Centigrade, and pressure is at or near atmospheric.

### Example 1

### Expression of the Fusion Protein of the Invention

The fusion proteins of the invention consist of a modified hetero-dimeric ubiquitin as targeting domain and at least one interferon, preferably interferon-alpha. In one embodiment, fusion proteins were produced as inclusion bodies in E. coli and purified after in vitro refolding. By way of characterization, the obtained fusion protein preparations were analyzed for purity and homogeneity. IFN-alpha activity in cell culture, IFN-alpha receptor binding activity, affinity for target protein ED-B, selectivity, and specific binding in cell culture were tested. Most preferred are fusion proteins with the parts of the fusion protein from the N-terminus to the C-terminus arranged as follows:
modified hetero-dimeric ubiquitin protein-linker-IFN, preferably IFN-α, or biologically active mutein thereof. Particularly preferred is IFN-alpha, more preferred IFN-alpha 2, and even more preferred IFN-alpha 2a or IFN-alpha 2b.

### Step 1: Production of a Vector for Cloning of Fusion Proteins

As vector for cloning of fusion proteins, commercially available vectors (e.g., pET20b by Invitrogen) or proprietary expression vectors (Scil Proteins GmbH, pSCIL008b, see WO 05/061716) were modified by insertion of coding sequences for IFN-alpha. The IFN-alpha sequence was amplified via PCR. Unique restriction sites were introduced into the resulting expression plasmids in order to facilitate the insertion of modified ubiquitin sequences.

### Step 2: Cloning of Modified Hetero-Dimeric Ubiquitin-Based ED-B-Fusion Proteins

For the production of the fusion proteins of ED-B-binding modified ubiquitin-based variants and IFN-alpha, the sequence of interest coding for ED-B-binding modified ubiquitin was amplified from a plasmid template by PCR according to standard procedures, and inserted into the expression plasmids described in step 1. DNA sequence analyses confirmed the correct sequences of the expression vectors encoding the fusion proteins.

### Step 3. Expression of Modified Hetero-Dimeric Ubiquitin-Based ED-B Fusion Proteins

Fusion proteins were produced in E. coli and isolated in the form of inclusion bodies. For expression of the fusion proteins, the clones were cultivated and grown by fed-batch fermentation in complex media containing the appropriate antibiotics corresponding to the respective expression vectors. Expression was induced by adding IPTG. After 2-4 h of induction, microbial cells were harvested, suspended, and disrupted by high pressure dispersion in a French press. The insoluble fraction was collected and inclusion bodies containing the expressed proteins were isolated by standard washing protocols.

### Example 2

### In Vitro Refolding and Purification of Ubiquitin-Based-IFN-Alpha Fusion Proteins

Active fusion proteins were prepared by in vitro refolding at a temperature of 4 degrees centigrade after rapid dilution of inclusion body material solubilized in 6 M guanidinium chloride, and purified by a series of chromatographic steps. At least two chromatographic steps are required for purification. These chromatographic steps included a capture step on a hydrophobic interaction chromatography on, e.g. Octyl-FF, and/or an ion exchange chromatography on, e.g. Q Sepharose HP. In all cases, fractions were analyzed by SDS-PAGE and analytical HPLC with respect to their purity. Suitable fractions were pooled and analyzed for homogeneity and activity by a series of methods including, e.g., rpHPLC, SE-HPLC, analytical affinity interaction chromatography, and surface plasmon resonance-based interaction analysis. For fusion protein 1354-A8-IFN, yields of up to 150 mg active fusion protein per liter expression culture from fed-batch fermentation were obtained. Yields obtained for other fusion variants were comparable. For non-targeting UB2-IFN, yields of up to 690 mg active fusion protein per liter expression culture from fed-batch fermentation were obtained.

### Example 3

### Binding of the Fusion Protein to an Interferon Receptor

Binding of the interferon α/β receptor to the interferon moiety of the fusion protein was probed in an ELISA setup. A non-neutralizing interferon-antibody was coated to Nunc microwell plates in a concentration of 2-10 μg/ml overnight at 4° C. After washing the plate with PBS, pH 7.4, the wells were blocked with casein blocking solution in PBS for 2 hours at room temperature. After washing the wells with PBST the fusion protein was applied to the wells in appropriate concentrations for 1 hour at room temperature. The wells were washed three times with PBST. The interferon α/β receptor is fused to the Fc portion of human IgG. This chimera was applied to the wells in a concentration of 0.345 μg/ml and incubated for 1 hour at room temperature. After washing the wells with PBST, a Hrp-conjugate of Fc-specific anti human IgG was applied in an appropriate dilution (for example, 1:10000) in PBST. The plate was washed three times with 300 μl buffer PBST/well. 50 μl TMB substrate solution (KEM-EN-Tec) was added to each well and was incubated. The reaction was stopped by adding 0.2 M H₂SO₄ per well. The ELISA plates were read out using the TECAN Sunrise ELISA-Reader. The photometric absorbance measurements were done at 450 nm using 620 nm as a reference wavelength. FIG. 5 shows clearly the very high affinity binding interferon αβ receptor to the interferon moiety of the fusion protein (SEQ ID NO: 55) with an apparent KD value of 16.7 nM. This result proves that the IFN-alpha receptor binding activity of the fusion protein is not impaired.

### Example 4

### Binding Analysis of the Fusion Protein to Human ED-B by Biacore Assays

Different concentrations of the fusion protein (1041-D11-IFN) were analyzed (for example, 0-500 nM) for binding to ED-B immobilized on a CM5-chip (Biacore) using methods known to those skilled in the art. The obtained data were processed via the BIAevaluation software and 1:1-Langmuir-fitting. The K_{D} was 7.25 nM. The kinetic binding constants were kₒₙ=4.16*10⁵ M⁻¹s⁻¹; k_{off}=3.01*10⁻³ s⁻¹.

### Example 5

### Freeze/Thaw Experiment

ED-B ELISA: Increasing amounts of purified protein applied to NUNC-medisorp plates coated with human ED-B and NGF served as negative control. Antigen coating with 1 to 2.5 μg/ml per well was performed at 4° C. overnight. After washing the plates with PBS, 0.1% Tween 20 pH 7.4 (PBST) the wells were blocked using blocking solution (PBS pH 7.4; 3% BSA; 0.5% Tween 20) at room temperature for 2 h. Wells were washed again three times with PBST. Different concentrations of fusion protein were then incubated in the wells at RT for 1 h. After washing the wells with PBST, the anti-Ubi fab fragment (a-Ubi-Fab) POD conjugate was applied in an appropriate dilution (for example, 1:6500) in PBST. The plate was washed three times with 300 μl buffer PBST/well. 50 μl TMB substrate solution (KEM-EN-Tec) was added to each well and was incubated. The reaction was stopped by adding 0.2 M H₂SO₄ per well. The ELISA plates were read out using the TECAN Sunrise ELISA-Reader. The photometric absorbance measurements were done at 450 nm using 620 nm as a reference wavelength.

Table 3 summarizes functionality of the interferon-domain as well as the affinity binding of the fusion protein of the invention after multiple freeze/thaw cycles at −80° C. Sample aliquots were frozen up to three times and thawed at room temperature prior to analysis. Functionality of the interferon-domain was determined via concentration dependent ELISA as described in example 1. Binding of the fusion protein to human ED-B was assayed by a concentration dependent ELISA, too. There was no detectable decrease after the described conditions, neither for the functionality of the interferon-domain nor for the affinity of the binding site of the described fusion protein.

TABLE 3

| | Functionality of the | Binding affinity |
|---|---|---|
| | interferon-domain | of the fusion protein |
| No of freeze/thaw steps | [KD value] | [KD value] |
| Initial (0x) | 27 nM | 15.5 nM |
| 1x freeze/thaw | 19 nM | 16.2 nM |
| 2x freeze/thaw | 23 nM | 20.6 nM |
| 3x freeze/thaw | 18 nM | 14.6 nM |

### Example 6

### Specific ED-B-Binding of Fusion Proteins of the Invention on Fibroblast Cells

ED-B is expressed in tumors and the matrix on embryonic cells. Wi38 cells are normal human embryonic lung fibroblast cells having high expression of ED-B. Normal adult human dermal fibroblast cell line expressing a low level of ED-B was used as negative control. To analyze in vitro binding of the fusion proteins of the invention to ED-B, fusion proteins 1041-D11-IFN, 1255-B9-IFN, 1353-H6-IFN, 1354-A8_F63P-IFN and UB2-IFN (control) were investigated on fixed Wi38 cells.

For staining experiments, 60,000 cells/ml were cultivated, followed by fixation with absolute cold methanol, blocking with 5% horse serum, and incubation with 50 nM of the fusion proteins 1041-D11-IFN, 1255-B9-IFN, 1353-H6-IFN, 1354-A8-IFN and UB2-IFN or PBS as control. The binding of fusion proteins to the cells was detected with a direct FITC labeled anti-IFN-antibody (PBL, 21112-3). Nuclei were stained with DAPI.

The analysis clearly shows that IFN-alpha-fused cancer binding proteins bind to vital Wi38 cells with high specificity to ED-B containing extracellular matrix (see FIG. 6). Especially 1353-H6-IFN and 1354-A8-IFN show a strong binding with 50 nM to Wi38-cells, but a negative staining to normal fibroblast cells. An unspecific binding of the IFN-alpha-antibody can be excluded by PBS control and the staining with the non binding protein UB2-IFN. The negative control cell type NHDF is a primary normal fibroblast cell line, which express low levels of ED-B-fibronectin. No binding on NHDF-cells can be observed.

### Example 7

### Specificity of ED-B-Binding of the Fusion Proteins on F9-Tumor Slices

ED-B accumulates around neovascular structures, like tumor blood vessels (Tarli et al., 1999, Blood 94: 192-198). Different concentrations of fusion proteins of the invention were compared with respect to ED-B-binding on F9 teratocarcinoma slices. Slices of a thickness of 6 μm were fixed with ice cold absolute Acetone. After blocking with 5% horse serum, slices were incubated with 10 and 50 nM 1255-B9-IFN (SEQ ID NO: 56), 1041-D11-IFN (SEQ ID NO: 55), 1353-H6-IFN (SEQ ID NO: 82), 1354-A8-IFN (SEQ ID NO: 84), and as control the non-targeting protein UB2-IFN (SEQ ID NO: 89) and PBS, respectively. Fusion proteins were detected with a direct FITC-labeled anti-IFN-alpha-antibody (PBL, 21112-3). CD31 (PECAM-1) is a widely used endothelial cell marker. Vessels were stained by use of a 10 μg/ml anti-mouse CD31-antibody (Abcam, ab56299) and an Alexa594-conjugated secondary antibody.

FIG. 7 shows a specific binding of 1255-B9-IFN, 1353-H6-IFN, 1354-A8-IFN and 1041-D11-IFN at 10 nM and 50 nM concentrations on F9-tumor tissue. No unspecific staining of anti-IFN-alpha-antibody was detected with the non-targeting fusion protein UB2-IFN and in the control slice (PBS). All binding proteins fused to IFN-alpha provide a predominant vessel association. The results clearly show the high specific targeting function of fusion protein.

### Example 8

### Activity-Assay of the Effector Domain of Different Fusion Proteins of the Invention

To analyze the physiological IFN-alpha activity of fusion proteins of the invention, an ISRE-Reporter Gene Assay was established. IFN-alpha is capable of inducing interferon-stimulated genes (ISGs), like ISG54. ISG54 contains a cis-acting element (TAGTTTCACTTTCCC, SEQ ID NO: 86) in its promoter, which is responsible for the inducible expression of the gene. This element is referred to as ISRE-element (IFN-stimulated response element). Five tandem copies of the ISRE element were inserted upstream of the basic promoter element (TATA box) and luciferase gene of pGL4.27-Luc2 plasmid (Promega). Hela-cells, a cervix carcinoma cell line, were transfected and a cell pool was sustained by selection with Hygromycin. To monitor the IFN-alpha activity of IFN-alphafusion proteins of the invention the reporter cells were used for an ISRE-Reporter Gene Assay.

The cells were resuspended in suitable medium containing 10% FCS. A cell suspension of a density of 3×10⁵ cells/ml in medium containing 5% FCS has been seeded into a white 96 well cell culture plate. After 24 h, the cells were treated with a range of concentrations of fusion proteins (e.g. between 3×10⁻¹⁰ and 4.6×10⁻¹⁴ M). The metabolic activity was measured by ONE-Glo™ Luciferase substrate (Promega). Each testing of fusion proteins of the invention was paralleled by testing a dose range of recombinant human IFN-alpha 2b (Biomol) to validate the assay. The quantitative evaluation is based on the relative potency against an IFN-alpha 2b standard by parallel line method with PLA2.0 software. Potency has been determined in triplicates. The fusion proteins have a potency of 7.9-22.7% to the internal standard. The potency of IFN-alpha 2b should be in a range of 100%+/−20% and the mean EC50 calculated by 4 parameter logistic method is 2.3+/−0.4 μM (see Table 4).

Table 4 summarizes the results of affinity and activity analysis of the fusion proteins.

TABLE 4
Analysis of the fusion proteins

| | | Specific activity | Specific |
|---|---|---|---|
| | Specific affinity | in ISRE reporter | activity in |
| | against target ED-B | gene assay | IFN receptor |
| | [K_{D} via Biacore] | [% potency] | ELISA [K_{D}] |
| 1041-D11-IFN | 7 nM | 18.7 ± 1.98 | 10.2 nM |
| (SEQ ID NO: 55) | | | |
| 1255-B9-IFN | 76 pM | 9.5 ± 1.56 | 7.6 nM |
| (SEQ ID NO: 56) | | | |
| 1353-H6-IFN | 26 pM | 7.9 ± 0.71 | 17.1 nM |
| (SEQ ID NO: 82) | | | |
| 1354-A8-IFN | 139 pM | 22.7 ± 1.00 | 22.0 nM |
| (SEQ ID NO: 84) | | | |

### SEQUENCE LISTING

### Free Text Information

The sequences according to SEQ ID NOs: 1, 2, and 8-18 shown in the attached sequence listing do not contain any free text information. Nevertheless, short explanations are presented below also for these sequences.
SEQ ID NO: 1: ubiquitin
SEQ ID NO: 2: extradomain B (ED-B) of fibronectin
SEQ ID NO: 3: linker sequence
SEQ ID NO: 4: linker sequence
SEQ ID NO: 5: linker sequence
SEQ ID NO: 6: linker sequence
SEQ ID NO: 7: linker sequence
SEQ ID NO: 8: IFN-alpha-2a, human
SEQ ID NO: 9: IFN-alpha-2a with signal peptide, human
SEQ ID NO: 10: IFN-alpha-2b, human
SEQ ID NO: 11: IFN-alpha-2c, human
SEQ ID NO: 12: IFN-alpha-6, human
SEQ ID NO: 13: IFN-alpha-14, human
SEQ ID NO: 14: IFN-alpha-4, human
SEQ ID NO: 15: IFN-alpha-5, human
SEQ ID NO: 16: IFN-alpha-2, mouse

### SEQ ID NO: 17: IFN-alpha-1, rat

SEQ ID NO: 18: IFN-alpha, rabbit
SEQ ID NO: 19: 1041-D11, modified hetero-dimeric ubiquitin protein
SEQ ID NO: 20: 1255-B9, modified hetero-dimeric ubiquitin protein
SEQ ID NO: 21: 1255-B10, modified hetero-dimeric ubiquitin protein
SEQ ID NO: 22 1247-G11, modified hetero-dimeric ubiquitin protein
SEQ ID NO: 23: 1255-G12, modified hetero-dimeric ubiquitin protein
SEQ ID NO: 24: 1247-F8, modified hetero-dimeric ubiquitin protein
SEQ ID NO: 25: 1237-B10, modified hetero-dimeric ubiquitin protein
SEQ ID NO: 26: 1237-H4, modified hetero-dimeric ubiquitin protein
SEQ ID NO: 27: 1239-B10, modified hetero-dimeric ubiquitin protein
SEQ ID NO: 28: 1246-H5, modified hetero-dimeric ubiquitin protein
SEQ ID NO: 29: 1247-G1, modified hetero-dimeric ubiquitin protein
SEQ ID NO: 30: 1247-H2, modified hetero-dimeric ubiquitin protein
SEQ ID NO: 31: 1248-E1, modified hetero-dimeric ubiquitin protein
SEQ ID NO: 32: 1249-E5, modified hetero-dimeric ubiquitin protein
SEQ ID NO: 33: 1253-A11, modified hetero-dimeric ubiquitin protein
SEQ ID NO: 34: 1255-A8, modified hetero-dimeric ubiquitin protein
SEQ ID NO: 35: 1255-G3, modified hetero-dimeric ubiquitin protein
SEQ ID NO: 36: 1255-H3, modified hetero-dimeric ubiquitin protein
SEQ ID NO: 37: fusion protein IFN-1041-D11
SEQ ID NO: 38: fusion protein IFN-1255-B9
SEQ ID NO: 39: fusion protein IFN-1255-B10
SEQ ID NO: 40: fusion protein IFN-1247-G11
SEQ ID NO: 41: fusion protein IFN-1255-G12
SEQ ID NO: 42: fusion protein IFN-1247-F8
SEQ ID NO: 43: fusion protein IFN-1237-B10
SEQ ID NO: 44: fusion protein IFN-1237-H4
SEQ ID NO: 45: fusion protein IFN-1239-B10
SEQ ID NO: 46: fusion protein IFN-1246-H5
SEQ ID NO: 47: fusion protein IFN-1247-G1
SEQ ID NO: 48: fusion protein IFN-1247-H2
SEQ ID NO: 49: fusion protein IFN-1248-E1
SEQ ID NO: 50: fusion protein IFN-1249-E5
SEQ ID NO: 51: fusion protein IFN-1253-A11
SEQ ID NO: 52: fusion protein IFN-1255-A8
SEQ ID NO: 53: fusion protein IFN-1255-G3
SEQ ID NO: 54: fusion protein IFN-1255-H3
SEQ ID NO: 55: fusion protein 1041-D11-IFN
SEQ ID NO: 56: fusion protein 1255-B9-IFN
SEQ ID NO: 57: fusion protein 1255-B10-IFN
SEQ ID NO: 58: fusion protein 1247-G11-IFN
SEQ ID NO: 59: fusion protein 1255-G12-IFN
SEQ ID NO: 60: fusion protein 1247-F8-IFN
SEQ ID NO: 61: fusion protein 1237-B 10-IFN
SEQ ID NO: 62: fusion protein 1237-H4-IFN
SEQ ID NO: 63: fusion protein 1239-B10-IFN
SEQ ID NO: 64: fusion protein 1246-H5-IFN
SEQ ID NO: 65: fusion protein 1247-G1-IFN
SEQ ID NO: 66: fusion protein 1247-H2-IFN
SEQ ID NO: 67: fusion protein 1248-E1-IFN
SEQ ID NO: 68: fusion protein 1249-E5-IFN
SEQ ID NO: 69: fusion protein 1253-A11-IFN
SEQ ID NO: 70: fusion protein 1255-A8-IFN
SEQ ID NO: 71: fusion protein 1255-G3-IFN
SEQ ID NO: 72: fusion protein 1255-H3-IFN
SEQ ID NO: 73: linker sequence RIG
SEQ ID NO: 74: 1353-H6, modified hetero-dimeric ubiquitin protein
SEQ ID NO: 75: 1351-E9, modified hetero-dimeric ubiquitin protein
SEQ ID NO: 76: 1354-A8, modified hetero-dimeric ubiquitin protein
SEQ ID NO: 77: 1351-E9_F63P, modified hetero-dimeric ubiquitin protein
SEQ ID NO: 78: fusion protein IFN-1353-H6
SEQ ID NO: 79: fusion protein IFN-1351-E9
SEQ ID NO: 80: fusion protein IFN-1354-A8_F63P
SEQ ID NO: 81: fusion protein IFN-1351-E9_F63P
SEQ ID NO: 82: fusion protein 1353-H6-IFN
SEQ ID NO: 83: fusion protein 1351-E9-IFN
SEQ ID NO: 84: fusion protein 1354-A8-IFN
SEQ ID NO: 85: fusion protein 1351-E9_F63P-IFN
SEQ ID NO: 86 cis-acting element
SEQ ID NO: 87 basic linker sequence
SEQ ID NO: 88 basic linker sequence
SEQ ID NO: 89 fusion protein UB2-IFN
SEQ ID NO: 90 fusion protein IFN-UB2
SEQ ID NO: 91: ubiquitin mutein (F45W/G75A/G76A), start sequence for mutagenesis

1. A fusion protein comprising the following parts:
(i) an interferon or a biologically active mutein thereof;
(ii) a modified hetero-dimeric ubiquitin protein that is capable of binding to a target molecule; and
(iii) optionally a linker.

2. The fusion protein according to claim 1, wherein the interferon is interferon-alpha (IFN-α) or interferon-beta (IFN-β), preferably human interferon-alpha or interferon-beta.

3. The fusion protein according to claim 2, wherein the IFN-α is selected from the group consisting of IFN-α 2a, IFN-α 2b, IFN-α 2c, IFN-α 6, IFN-α 14, IFN-α 4, IFN-α 5, and biologically active muteins of any of these.

4. The fusion protein according to claim 1, wherein the modified hetero-dimeric ubiquitin protein has a specific binding affinity to the target molecule of Kd≦10⁻⁷.

5. The fusion protein according to claim 1, wherein the modified hetero-dimeric ubiquitin protein comprises two monomeric ubiquitin units linked together in a head-to-tail arrangement.

6. The fusion protein according to claim 1, wherein each modified monomeric ubiquitin unit has an amino acid sequence identity of at least 80% to the amino acid sequence defined by SEQ ID NO: 1 or to the amino acid sequence defined by SEQ ID NO: 91.

7. The fusion protein according to claim 1, wherein each monomeric ubiquitin unit in said modified hetero-dimeric ubiquitin protein is modified independently from the modifications in the other monomeric ubiquitin unit by substitutions of 1-8 amino acids in regions 2-8 and/or 62-68 of SEQ ID NO: 1 or SEQ ID NO: 91.

8. The fusion protein according to claim 1, wherein the substitutions in the monomeric ubiquitin units comprise
(1) in the first monomeric unit: substitutions at least in one or more positions selected from the group of amino acid positions 6, 8, 63, 64, 65, and 66; and in the second monomeric unit: substitutions at least in one or more positions selected from the group of amino acid positions 6, 8, 62, 63, 64, 65, and 66; optionally additionally 2, or
(2) in the first monomeric unit: substitutions at least in one or more positions selected from the group of amino acid positions 2, 4, 6, 62, 63, 64, 65, and 66; and in the second monomeric unit: substitutions at least in one or more positions selected from the group of amino acid positions 6, 8, 62, 63, 64, and 66; optionally additionally 65,
and optionally further modifications, preferably substitutions of other amino acids.

9. The fusion protein according to claim 1, wherein from 1 to 7 amino acids are additionally modified in the modified hetero-dimeric ubiquitin protein.

10. The fusion protein according to claim 9, wherein said from 1 to 7 additionally modified amino acids are selected from one or more of the amino acids in positions 2, 4, 8, 33, 36, 38, 62, 44, 70, and 71 of the first monomeric ubiquitin unit and in positions 2, 10, 16, 34, 36, 44, 51, 53, 65, 70, and 71 of the second monomeric ubiquitin unit.

11. The fusion protein according to claim 1, wherein the linker is absent and the IFN, preferably IFN-α, and the modified hetero-dimeric ubiquitin protein are directly fused to each other.

12. The fusion protein according to claim 1, wherein the IFN, preferably IFN-α, or the biologically active mutein thereof is positioned C-terminally to the modified hetero-dimeric ubiquitin protein.

13. The fusion protein according to claim 1, wherein the IFN, preferably IFN-α, or the biologically active mutein thereof is positioned N-terminally to the modified hetero-dimeric ubiquitin protein.

14. The fusion protein according to claim 1, wherein the linker is present and the IFN, preferably IFN-α, or the biologically active mutein thereof and the modified hetero-dimeric ubiquitin protein are connected via the linker.

15. The fusion protein according to claim 14, wherein the order of the parts of the fusion protein from the N-terminus to the C-terminus is as follows: modified hetero-dimeric ubiquitin protein-linker-IFN, preferably IFN-α, or biologically active mutein thereof.

16. The fusion protein according to claim 14, wherein the order of the parts of the fusion protein from the N-terminus to the C-terminus is as follows: IFN, preferably IFN-α, or biologically active mutein thereof -linker modified hetero-dimeric ubiquitin protein.

17. The fusion protein according to claim 14, wherein said linker comprises an amino acid sequence selected from the following amino acid sequences: GIG (SEQ ID NO: 3), RIG (SEQ ID NO: 73), SGGGG (SEQ ID NO: 4), SGGGGIG (SEQ ID NO: 5), SGGGGSGGGGIG (SEQ ID NO: 6), SGGGGSGGGG (SEQ ID NO: 7), SG, (SGGG (SEQ ID NO: 88))ₙ with n being between 1 and 10, and (GGGS (SEQ ID NO: 87))ₙ with n being between 1 and 10.

18. The fusion protein according to claim 1, wherein the target molecule is the extradomain B (ED-B) of fibronectin.

19. The fusion protein according to claim 1, wherein the modified hetero-dimeric ubiquitin protein comprises an amino acid sequence selected from the group consisting of: SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 74, SEQ ID NO: 75, SEQ ID NO: 76, SEQ ID NO: 77, and an amino acid sequence that exhibits at least 90% sequence identity to one or more of the amino acid sequences according to SEQ ID NOs: 19 to 36 or to SEQ ID NOs: 74 to 77.

20. The fusion protein according to claim 1, wherein the fusion protein comprises an amino acid sequence selected from the group consisting of: SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, and an amino acid sequence that exhibits at least 90% sequence identity to one or more of the amino acid sequences according to SEQ ID NOs: 37 to 72 or to SEQ ID NOs: 78 to 85.

21. The fusion protein according to claim 1, for use in medicine.

22. The fusion protein according to claim 1, for use in the treatment of cancer or infectious diseases.

23. The fusion protein according to claim 1, for use in medicine, preferably for use in the treatment of cancer, wherein the fusion protein is for administration in combination with a cancer therapeutic agent.

24. The fusion protein according to claim 1, wherein the cancer therapeutic agent is selected from the group consisting of CHOP, vinblastin, cytarabin, bevacizumab, tumor vaccines, radiopharmaceuticals, nanoparticular formulations of cytostatics and others, preferably selected from the group consisting of bevacizumab, CHOP, and vinblastin.

25. A polynucleotide encoding the fusion protein as defined in claim 1.

26. A vector comprising the polynucleotide of claim 25.

27. A host cell comprising
a fusion protein as defined in claim 1.

28. A pharmaceutical composition comprising
a fusion protein as defined in claim 1
and further comprising a pharmaceutically acceptable carrier.

29. The pharmaceutical composition of claim 28, further comprising one or more cancer therapeutic agents, selected from the group consisting of CHOP, vinblastin, cytarabin, bevacizumab, tumor vaccines, radiopharmaceuticals, nanoparticular formulations of cytostatics and others, preferably selected from the group consisting of bevacizumab, CHOP, and vinblastin.

30. The pharmaceutical composition of claim 28 or which is in the form of a combined preparation or in the form of a kit of parts.

31. A method for generating a fusion protein according to claim 1, said method comprising:
(a) providing a population of differently modified dimeric ubiquitin proteins originating from monomeric ubiquitin proteins, said population comprising dimeric ubiquitin proteins comprising two modified ubiquitin monomers linked together, preferably in a head-to-tail arrangement, wherein each monomer of said dimeric protein is differently modified by substitutions of 1-8 amino acids of SEQ ID NO: 1 or SEQ ID NO: 91;
(b) providing a target molecule as potential ligand;
(c) contacting said population of differently modified proteins with said target molecule;
(d) identifying a modified dimeric ubiquitin protein by a screening process, wherein said modified dimeric ubiquitin protein binds to said target molecule with a specific binding affinity of Kd≦10⁻⁷ M;
(e) isolating said modified dimeric ubiquitin protein with said binding affinity; and
(f) fusing IFN, preferably IFN-α, or a biologically active mutein thereof to the modified dimeric ubiquitin protein obtained in step e).

32. The method of claim 31, wherein the target molecule is the extradomain B (ED-B) of fibronectin.

33. A method for preparing a fusion protein according to claim 1, said method comprising:
(a) preparing a nucleic acid encoding a fusion protein according to claim 1;
(b) introducing said nucleic acid into an expression vector;
(c) introducing said expression vector into a host cell;
(d) cultivating the host cell;
(e) subjecting the host cell to culturing conditions under which a fusion protein is expressed from said vector, thereby producing a fusion protein according to claim 1; and
(f) optionally enriching or isolating the fusion protein produced in step (e).

34. The method of claim 33, wherein the fusion protein produced in step (e) is in the form of inclusion bodies.

35. The method of claim 34, further comprising:
(g) isolating the inclusion bodies;
(h) solubilizing said inclusion bodies, thereby obtaining soluble fusion proteins, and
(i) further purifying the soluble fusion proteins obtained in the preceding step by at least two chromatographic steps.
